(19) United States
(12) Patent Application Publication
(10) Pub. No.: US 2025/0027069 A1
(43) Pub. Date: Jan. 23, 2025

(54) ENGINEERED RUBISCO ENZYME COMPLEXES

(71) Applicant: Cornell University, Ithaca, NY (US)

(72) Inventors: Myat T. LIN, Hasbrouck Heights, NJ (US); Maureen HANSON, Ithaca, NY (US)

(21) Appl. No.: 18/707,464

(22) PCT Filed: Nov. 8, 2022
(86) PCT No.: PCT/US2022/079449

§ 371 Date: May 3, 2024

Related U.S. Application Data
(60) Provisional application No. 63/276,980, filed on Nov. 8, 2021.

(51) Int. Cl. C12N 9/88; C12N 15/82

(52) U.S. Cl. CPC C12N 9/88; C12N 15/8269; C12Y 401/01039

(57) ABSTRACT

Provided herein are genetically engineered Rubisco enzymes and plants comprising the same. In one aspect, the disclosure features a genetically engineered plant comprising a Rubisco large subunit (LSU) comprising L2251 and K429Q amino acid substitution mutations, wherein the amino acid substitution mutations are numbered relative to the LSU of Nicotiana tabacum; and a Rubisco small subunit (SSU comprising N8G, V301, and E88Q amino acid substitution mutations, wherein the amino acid substitution mutations are numbered relative to the S-T2 subunit of Nicotiana tabacum.

### CROSS-REFERENCE TO RELATED APPLICATIONS

This application claims benefit of U.S. Provisional Application No. 63/276,980, filed Nov. 8, 2021, the contents of which are incorporated herein by reference in their entirety.

### GOVERNMENT FUNDING

This work was supported at least in part by grant no. DE-SC0020142 awarded by the Department of Energy. The government has certain rights in the invention.

### SEQUENCE LISTING

The instant application contains a Sequence Listing which has been submitted electronically in XML format and is hereby incorporated by reference in its entirety. Said XML copy, created on Nov. 7, 2022, is named 50341-035WO2_Sequence_Listing_11_7_22 and is 51,117 bytes in size.

### FIELD OF THE INVENTION

Provided herein are genetically engineered Rubisco enzymes and plants comprising the same.

### BACKGROUND

Plants and photosynthetic organisms possess a remarkably inefficient enzyme named Rubisco that fixes atmospheric CO₂ into organic compounds. There is a need in the art for improved Rubisco enzymes, e.g., to improve photosynthesis in plants and/or to help plants adapt to anthropogenic climate change.

### SUMMARY OF THE INVENTION

In one aspect, the disclosure features a genetically engineered plant comprising (a) a Rubisco large subunit (LSU) comprising L225I and K429Q amino acid substitution mutations, wherein the amino acid substitution mutations are numbered relative to the LSU of Nicotiana tabacum (SEQ ID NO: 43); and (b) a Rubisco small subunit (SSU) comprising N8G, V30I, and E88Q amino acid substitution mutations, wherein the amino acid substitution mutations are numbered relative to the S-T2 subunit of Nicotiana tabacum (SEQ ID NO: 44).

In some aspects, the Rubisco LSU comprises an amino acid sequence having at least 80%, 85%, 90%, 95%, 98%, or 99% sequence identity to SEQ ID NO: 1. In some aspects, the Rubisco LSU comprises the amino acid sequence of SEQ ID NO: 1.

In some aspects, the Rubisco SSU comprises an amino acid sequence having at least 80%, 85%, 90%, 95%, 98%, or 99% sequence identity to SEQ ID NO: 20. In some aspects, the Rubisco SSU comprises the amino acid sequence of SEQ ID NO: 20.

In another aspect, the disclosure features a genetically engineered plant comprising (a) a Rubisco LSU comprising V145I, L225I, and K429Q amino acid substitution mutations, wherein the amino acid substitution mutations are numbered relative to the LSU of Nicotiana tabacum (SEQ ID NO: 43); and (b) a Rubisco SSU comprising N8G, V30I, and E88Q amino acid substitution mutations, wherein the amino acid substitution mutations are numbered relative to the S-T2 subunit of Nicotiana tabacum (SEQ ID NO: 44).

In some aspects, the Rubisco LSU comprises an amino acid sequence having at least 80%, 85%, 90%, 95%, 98%, or 99% sequence identity to SEQ ID NO: 2. In some aspects, the Rubisco LSU comprises the amino acid sequence of SEQ ID NO: 1.

In some aspects, the Rubisco SSU comprises an amino acid sequence having at least 80%, 85%, 90%, 95%, 98%, or 99% sequence identity to SEQ ID NO: 20. In some aspects, the Rubisco SSU comprises the amino acid sequence of SEQ ID NO: 20.

In another aspect, the disclosure features a genetically engineered plant comprising (a) a Rubisco LSU comprising L225I and K429Q amino acid substitution mutations, wherein the amino acid substitution mutations are numbered relative to the LSU of Nicotiana tabacum (SEQ ID NO: 43); and (b) a Rubisco SSU comprising N8G, K9M, E23D, R28K, V30I, and E88Q amino acid substitution mutations, wherein the amino acid substitution mutations are numbered relative to the S-T2 subunit of Nicotiana tabacum (SEQ ID NO: 44).

In some aspects, the Rubisco LSU comprises an amino acid sequence having at least 80%, 85%, 90%, 95%, 98%, or 99% sequence identity to SEQ ID NO: 1. In some aspects, the Rubisco LSU comprises the amino acid sequence of SEQ ID NO: 1.

In some aspects, the Rubisco SSU comprises an amino acid sequence having at least 80%, 85%, 90%, 95%, 98%, or 99% sequence identity to SEQ ID NO: 29. In some aspects, the Rubisco SSU comprises the amino acid sequence of SEQ ID NO: 29.

In another aspect, the disclosure features a genetically engineered plant comprising (a) a Rubisco LSU comprising V911, V145I, L225I, K429Q, E443D, C449S, V466R, A470E, V472M, and V474T amino acid substitution mutations, wherein the amino acid substitution mutations are numbered relative to the LSU of Nicotiana tabacum (SEQ ID NO: 43); and (b) a Rubisco SSU comprising N8G, K9M, S22T, E23D, R28K, V30I, N36K, N56H, E88Q, and Q96N amino acid substitution mutations, wherein the amino acid substitution mutations are numbered relative to the S-T2 subunit of Nicotiana tabacum (SEQ ID NO: 44).

In some aspects, the Rubisco LSU comprises an amino acid sequence having at least 80%, 85%, 90%, 95%, 98%, or 99% sequence identity to SEQ ID NO: 17. In some aspects, the Rubisco LSU comprises the amino acid sequence of SEQ ID NO: 17.

In some aspects, the Rubisco SSU comprises an amino acid sequence having at least 80%, 85%, 90%, 95%, 98%, or 99% sequence identity to SEQ ID NO: 39. In some aspects, the Rubisco SSU comprises the amino acid sequence of SEQ ID NO: 39.

In some aspects, the genetically engineered plant is a C3 plant. In some aspects, the C3 plant is a member of the Solanaceae, Poaceae, Fabaceae, Brassicaceae, Rosaceae, Euphorbiaceae, Amaranthaceae, or Malvaceae. In some aspects, the C3 plant is tobacco, tomato, potato, pepper, rice, wheat, barley, soybean, cowpea, peanut, cassava, spinach, or cotton.

In some aspects, the catalytic efficiency of Rubisco in the genetically engineered plant is increased relative to that of a plant not comprising the Rubisco LSU of (a) and the Rubisco SSU of (b).

In some aspects, the k_{cat} value of Rubisco in the genetically engineered plant is increased relative to that of a plant not comprising the Rubisco LSU of (a) and the Rubisco SSU of (b).

In some aspects, the ribulose-1,5-bisphosphate (RuBP) carboxylation rate of Rubisco in the genetically engineered plant is increased relative to that of a plant not comprising the Rubisco LSU of (a) and the Rubisco SSU of (b).

In some aspects, expression of one or more endogenous Rubisco LSU or SSU genes in the genetically engineered plant has been reduced or eliminated. In some aspects, the reduction or elimination of expression comprises use of antisense technology or gene editing.

In some aspects, the Rubisco LSU of (a) and/or the Rubisco SSU of (b) is introduced to the genetically engineered plant by chloroplast transformation.

In some aspects, the Rubisco LSU of (a) and/or the Rubisco SSU of (b) is introduced to the genetically engineered plant by nuclear transformation.

In another aspect, the disclosure features a genetically engineered plant comprising (a) a Rubisco LSU comprising L225I and K429Q amino acid substitution mutations, wherein the amino acid substitution mutations are numbered relative to the LSU of Nicotiana tabacum (SEQ ID NO: 43); and (b) a Rubisco SSU comprising N8G, K9M, E23D, R28K, V30I, K57R, and E88Q amino acid substitution mutations, wherein the amino acid substitution mutations are numbered relative to the S-T2 subunit of Nicotiana tabacum (SEQ ID NO: 44).

In some aspects, the Rubisco LSU comprises an amino acid sequence having at least 80%, 85%, 90%, 95%, 98%, or 99% sequence identity to SEQ ID NO: 1. In some aspects, the Rubisco LSU comprises the amino acid sequence of SEQ ID NO: 1.

In some aspects, the Rubisco SSU comprises an amino acid sequence having at least 80%, 85%, 90%, 95%, 98%, or 99% sequence identity to SEQ ID NO: 34. In some aspects, the Rubisco SSU comprises the amino acid sequence of SEQ ID NO: 34.

In another aspect, the disclosure features a genetically engineered plant comprising (a) a Rubisco LSU comprising an L225I amino acid substitution mutation, wherein the amino acid substitution mutation is numbered relative to the LSU of Nicotiana tabacum (SEQ ID NO: 43); and (b) a Rubisco SSU comprising K9M, E23D, R28K, V30I, K57R, and E88Q amino acid substitution mutations, wherein the amino acid substitution mutations are numbered relative to the S-T2 subunit of Nicotiana tabacum (SEQ ID NO: 44).

In some aspects, the Rubisco LSU comprises an amino acid sequence having at least 80%, 85%, 90%, 95%, 98%, or 99% sequence identity to SEQ ID NO: 4. In some aspects, the Rubisco LSU comprises the amino acid sequence of SEQ ID NO: 4.

In some aspects, the Rubisco SSU comprises an amino acid sequence having at least 80%, 85%, 90%, 95%, 98%, or 99% sequence identity to SEQ ID NO: 35. In some aspects, the Rubisco SSU comprises the amino acid sequence of SEQ ID NO: 35.

In another aspect, the disclosure features a genetically engineered plant comprising (a) a Rubisco LSU comprising L225I and K429Q amino acid substitution mutations, wherein the amino acid substitution mutations are numbered relative to the LSU of Nicotiana tabacum (SEQ ID NO: 43); and (b) a Rubisco SSU comprising K9M, E23D, R28K, V30I, K57R, and E88Q amino acid substitution mutations, wherein the amino acid substitution mutations are numbered relative to the S-T2 subunit of Nicotiana tabacum (SEQ ID NO: 44).

In some aspects, the Rubisco LSU comprises an amino acid sequence having at least 80%, 85%, 90%, 95%, 98%, or 99% sequence identity to SEQ ID NO: 1. In some aspects, the Rubisco LSU comprises the amino acid sequence of SEQ ID NO: 1.

In some aspects, the Rubisco SSU comprises an amino acid sequence having at least 80%, 85%, 90%, 95%, 98%, or 99% sequence identity to SEQ ID NO: 35. In some aspects, the Rubisco SSU comprises the amino acid sequence of SEQ ID NO: 35.

In another aspect, the disclosure features a genetically engineered plant comprising (a) a Rubisco LSU comprising any one of the sets of amino acid substitution mutations listed in Table 3, wherein the amino acid substitution mutations are numbered relative to the LSU of Nicotiana tabacum (SEQ ID NO: 43); and (b) a Rubisco SSU comprising any one of the sets of amino acid substitution mutations listed in Table 3, wherein the amino acid substitution mutations are numbered relative to the S-T2 subunit of Nicotiana tabacum (SEQ ID NO: 44).

In some embodiments, (a) the Rubisco LSU comprises an amino acid sequence having at least 80%, 85%, 90%, 95%, 98%, or 99% sequence identity to any one of SEQ ID NOs: 1-19; and/or (b) the Rubisco SSU comprises an amino acid sequence having at least 80%, 85%, 90%, 95%, 98%, or 99% sequence identity to any one of SEQ ID NOs: 20-42.

Other features and advantages of the invention will be apparent from the following Drawings, Detailed Description, and the Claims.

### BRIEF DESCRIPTION OF THE DRAWINGS

FIG. 1A is a schematic diagram showing a workflow for de novo assembly of Rubisco transcripts from RNA-Seq data. The workflow processes one SRA at a time by downloading it with SRA Toolkit, extracting the reads aligned to Rubisco subunit sequences with BBMap program, assembling them de novo with the Trinity program, and removing potential chimeric assemblies in two clean-up steps. Chimeras with gaps in read coverages of starting bases were identified and removed in the first clean-up step. More potential chimeras with long overlaps with other assemblies were removed in the second clean-up step. The steps automated with Python scripts are indicated with green arrows.

FIG. 1B is a set of Venn diagrams showing the numbers of unique L and S subunit protein sequences in Solanaceae assembled in the present study (“assembled from SRAs”) and previously available.

FIG. 2A is a simplified phylogenetic tree for Solanaceae L subunits obtained from Bayesian inference. The fossil-calibrated divergent times for three ancestral nodes (Morita et al., Plant Physiol., 164: 69-79, 2014) as well as the names of eight ancestral nodes selected in this study are indicated. The inset displays the history of atmospheric CO₂ levels estimated from sea surface pH (Spreitzer et al., Proc. Natl. Acad. Sci. U.S.A., 102: 17225-17230, 2005) with the arrows indicating periodic CO₂ reductions that likely resulted in evolution of C₄ photosynthesis in several other families.

FIG. 2B is a simplified phylogenetic tree for Solanaceae S subunits obtained from Bayesian inference. The names of eight ancestral nodes are indicated.

FIG. 2C is a summary of L and S subunits and Rubiscos predicted for different ancestral nodes of Solanaceae.

FIG. 3 is a bar graph showing the results of an initial screening of Ribulose 1,5-bisphosphate (RuBP) carboxylation rates from the indicated predicted ancestral Rubiscos. The RuBP carboxylation rates were measured at a saturating [CO₂] of 108 μM at 25° C. under N2 and normalized to the numbers of active sites. Each bar in the chart shows the ratio of the mean of two technical replicates from each sample to that from the tobacco Rubisco with S-T2 subunit expressed in E. coli. Carboxylation kinetics at 25° C. were measured for samples marked with * or ** (see FIGS. 4A, 4B, and 5). Native PAGE analysis was carried out for samples marked with t (see FIG. 6). Carboxylation kinetics at 30° C. and S_{C/O} at 25° C. were measured for samples marked with ** (see Table 4 and FIG. 7). WT: wild type.

FIG. 4A is a scatterplot for Michaelis-Menten constants for CO₂ in air (K_{M,air}) vs. catalytic turnover numbers (k_{cat}) at 25° C. (B) A scatterplot for catalytic efficiency (k_{cat}/K_{M,air}) vs. k_{cat} at 25° C. RuBP carboxylation rates were measured for 38 predicted ancestors, three tobacco Rubiscos with different S subunits, and seven native Rubiscos from leaf tissues at six [CO₂]s, and K_{M,air} and k_{cat} were obtained from nonlinear least square fitting to the classical Michaelis-Menton equation. The means of measurements from three (n=3) E. coli soluble extracts or tobacco leaf soluble extracts from each sample were plotted. The identities of native Rubiscos are as follows: Nb=Nicotiana benthamiana, Np=Nicandra physalodes, Nt=Nicotiana tabacum (Petit Havana), Ph=Petunia hybrida, SI=Solanum lycopersicum (M28), Ss=Solanum sarrachoides, and St=Solanum tuberosum (Russett Burbank). The SDs and P values compared to the tobacco enzyme are summarized in Table 5.

FIG. 4B is a scatterplot for k_{cat}/K_{M,air} versus k_{cat} at 25° C.

FIG. 5 is a set of box-and-whisker plots showing k_{cat}, K_{M,air}, and k_{cat}/K_{M,air} at 25° C. reported in the literature for Rubiscos from C₃ plants and C₄ plants (Flamholz et al., Biochemistry, 58: 3365-3376, 2019) and those measured in the present study from Solanaceous plants and predicted ancestral Rubiscos expressed from E. coli.

FIG. 6 is a photograph of an immunoblot showing the results of a native PAGE analysis of the indicated Rubisco complexes in the soluble extracts of tobacco leaf tissue and E. coli cultures. The immunoblot was performed with an antibody that recognizes form IB Rubisco.

FIG. 7A is a bar graph showing the CO₂/O₂ specificity factors (Scio) of the indicated predicted ancestral Rubiscos of Solanaceae. The specificity factors were measured at three [CO₂]/[O₂] ratios at 25° C., and the means and SDs of five or six (n) technical replicates are plotted. The P values compared to the measurements from the tobacco enzyme with L and S-S1 subunits were determined with two-tailed heteroscedastic t tests.

FIG. 7B is a set of box-and-whisker plots showing a comparison of S_{C/O} at 25° C. reported in the literature for Rubiscos from C₃ plants and C₄ plants (Flamholz et al., Biochemistry, 58: 3365-3376, 2019) and those measured in the present study for predicted ancestral Rubiscos expressed from E. coli.

FIG. 8 is a consensus tree of Solanaceae L subunits obtained from Bayesian inference with the MrBayes program. The posterior probabilities of the nodes are also indicated.

FIG. 9 is a consensus tree of Solanaceae S subunits obtained from Bayesian inference with the MrBayes program. The posterior probabilities of the nodes are also indicated.

FIG. 10 is a phylogenetic tree of Solanaceae L subunits obtained from Maximum likelihood with the RAxML program. The bootstrap value of each node is also indicated.

FIG. 11 is a phylogenetic tree of Solanaceae S subunits obtained from Maximum likelihood with the RAxML program. The bootstrap value of each node is also indicated.

### DETAILED DESCRIPTION OF THE INVENTION

### Definitions

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, sap (e.g., xylem sap and phloem sap), and various forms of cells and culture (e.g., single cells, protoplasts, embryos, and callus tissue). The plant tissue may be in a plant or in a plant organ, tissue, or cell culture. In addition, a plant may be genetically engineered to produce a heterologous protein or RNA, for example, of any of the pest control (e.g., biopesticide or biorepellent) compositions in the methods or compositions described herein.

The terms “Rubisco large subunit” and “Rubisco LSU,” as used herein, refer to any Rubisco LSU from any photosynthetic organism, including plants (e.g., C₃ plants), algae, and cyanobacteria, unless otherwise indicated. The term encompasses naturally occurring and engineered variants of the Rubisco LSU. The amino acid sequence of an exemplary Rubisco LSU from Nicotiana tabacum is provided as SEQ ID NO: 43. Minor sequence variations, especially conservative amino acid substitutions of the Rubisco LSU that do not affect Rubisco LSU function and/or activity, are also contemplated by the invention.

The terms “Rubisco small subunit” and “Rubisco SSU,” as used herein, refer to any Rubisco SSU from any photosynthetic organism (e.g., any Rubisco S-T2 subunit), including plants (e.g., C3 plants), algae, and cyanobacteria, unless otherwise indicated. The term encompasses naturally occurring and engineered variants of the Rubisco SSU. The amino acid sequence of an exemplary Rubisco SSU from Nicotiana tabacum is provided as SEQ ID NO: 44. Minor sequence variations, especially conservative amino acid substitutions of the Rubisco SSU that do not affect Rubisco SSU function and/or activity, are also contemplated by the invention.

### I. IMPROVED RUBISCO ENZYMES AND PLANTS COMPRISING THE SAME

Provided herein are engineered Rubisco enzymes having amino acid residues identified in predicted ancestral Rubisco enzymes in the family Solanaceae (Table 3). Also provided herein are plants that have been modified (e.g., genetically engineered) to comprise a Rubisco large subunit (LSU) and/or a Rubisco small subunit (SSU) comprising the residues identified in the predicted ancestral Rubisco enzymes. Sequences of the predicted ancestral Rubisco enzymes are provided below.

In one aspect, the disclosure features a Rubisco enzyme complex comprising (a) a Rubisco LSU comprising any one of the sets of amino acid substitution mutations listed in Table 3, wherein the amino acid substitution mutations are numbered relative to the LSU of Nicotiana tabacum (SEQ ID NO: 43); and (b) a Rubisco SSU comprising any one of the sets of amino acid substitution mutations listed in Table 3, wherein the amino acid substitution mutations are numbered relative to the S-T2 subunit of Nicotiana tabacum (SEQ ID NO: 44). In some embodiments, (a) the Rubisco LSU comprises an amino acid sequence having at least 80%, 85%, 90%, 95%, 98%, or 99% sequence identity to any one of SEQ ID NOs: 1-19 (e.g., comprises the amino acid sequence of any one of SEQ ID NOs: 1-19); and/or (b) the Rubisco SSU comprises an amino acid sequence having at least 80%, 85%, 90%, 95%, 98%, or 99% sequence identity to any one of SEQ ID NOs: 20-42 (e.g., comprises the amino acid sequence of any one of SEQ ID NOs: 20-42).

Further provided herein are genetic constructs (e.g., vectors) comprising any one of the Rubisco LSUs and/or SSUs provided herein, e.g., genetic constructs comprising (a) a nucleotide sequence encoding a Rubisco LSU comprising any one of the sets of amino acid substitution mutations listed in Table 3, wherein the amino acid substitution mutations are numbered relative to the LSU of Nicotiana tabacum (SEQ ID NO: 43) and/or (b) a nucleotide sequence encoding a Rubisco SSU comprising any one of the sets of amino acid substitution mutations listed in Table 3, wherein the amino acid substitution mutations are numbered relative to the S-T2 subunit of Nicotiana tabacum (SEQ ID NO: 44). In some embodiments, (a) the nucleotide sequence encodes a Rubisco LSU comprising an amino acid sequence having at least 80%, 85%, 90%, 95%, 98%, or 99% sequence identity to any one of SEQ ID NOs: 1-19 (e.g., encodes a Rubisco LSU comprising the amino acid sequence of any one of SEQ ID NOs: 1-19); and/or (b) the Rubisco SSU encodes a Rubisco LSU comprising an amino acid sequence having at least 80%, 85%, 90%, 95%, 98%, or 99% sequence identity to any one of SEQ ID NOs: 20-42 (e.g., encodes a Rubisco LSU comprising the amino acid sequence of any one of SEQ ID NOs: 20-42).

Further provided herein are genetically engineered plants, plant cells, plant parts, and plant seeds comprising any one of the genetic constructs and/or Rubisco LSUs and/or SSUs provided herein, e.g., genetically engineered plants comprising (a) a Rubisco LSU comprising any one of the sets of amino acid substitution mutations listed in Table 3, wherein the amino acid substitution mutations are numbered relative to the LSU of Nicotiana tabacum (SEQ ID NO: 43); and (b) a Rubisco SSU comprising any one of the sets of amino acid substitution mutations listed in Table 3, wherein the amino acid substitution mutations are numbered relative to the S-T2 subunit of Nicotiana tabacum (SEQ ID NO: 44). In some embodiments, (a) the Rubisco LSU comprises an amino acid sequence having at least 80%, 85%, 90%, 95%, 98%, or 99% sequence identity to any one of SEQ ID NOs: 1-19 (e.g., comprises the amino acid sequence of any one of SEQ ID NOs: 1-19); and/or (b) the Rubisco SSU comprises an amino acid sequence having at least 80%, 85%, 90%, 95%, 98%, or 99% sequence identity to any one of SEQ ID NOs: 20-42 (e.g., comprises the amino acid sequence of any one of SEQ ID NOs: 20-42).

For example, in some aspects, the disclosure features a genetically engineered plant, plant cell, plant parts, or plant seed comprising (a) a Rubisco LSU comprising any one of the sets of amino acid substitution mutations listed in Table 3, wherein the amino acid substitution mutations are numbered relative to the LSU of Nicotiana tabacum (SEQ ID NO: 43), or one or more constructs encoding the same; and (b) a Rubisco SSU comprising any one of the sets of amino acid substitution mutations listed in Table 3, wherein the amino acid substitution mutations are numbered relative to the S-T2 subunit of Nicotiana tabacum (SEQ ID NO: 44), or one or more constructs encoding the same. In some embodiments, (a) the Rubisco LSU comprises an amino acid sequence having at least 80%, 85%, 90%, 95%, 98%, or 99% sequence identity to any one of SEQ ID NOs: 1-19 (e.g., comprises the amino acid sequence of any one of SEQ ID NOs: 1-19); and/or (b) the Rubisco SSU comprises an amino acid sequence having at least 80%, 85%, 90%, 95%, 98%, or 99% sequence identity to any one of SEQ ID NOs: 20-42 (e.g., comprises the amino acid sequence of any one of SEQ ID NOs: 20-42).

Further provided herein are methods of making any one of the genetically engineered plants, plant cells, plant parts, or plant seeds described herein. In some embodiments, the Rubisco LSU of (a) and/or the Rubisco SSU of (b) is introduced to the genetically engineered plant by chloroplast transformation. In some embodiments, the Rubisco LSU of (a) and/or the Rubisco SSU of (b) is introduced to the genetically engineered plant by nuclear transformation. The genetically engineered plant may be modified using any method known in the art. Exemplary methods for modifying the L subunit, the S subunit, or both subunits simultaneously are provided, e.g., in Whitney et al., Proc. Natl. Acad. Sci. U.S.A., 108: 14688-14693, 2011; Lin et al., Plant J., 106: 876-887, 2021; Whitney et al., Proc. Nat. Acad. Sci. U.S.A., 112: 3564-3569, 2015; Donovan et al., Front. Genome Ed., 2: 605614, 2020; Matsumura et al., Mol. Plant, 13: 1570-1581, 2020; Zhang et al., Food Sci. Nutr., 8: 3479-3491, 2020; Gunn et al., Proc. Natl. Acad. Sci. U.S.A., 117: 25890-25896, 2020; Martin-Avila et al., Plant Cell, 32: 2898-2916, 2020; and Lin et al., Nature, 513: 547-550, 2014.

In some embodiments, expression of one or more endogenous Rubisco LSU or SSU genes in the genetically engineered plant (e.g., expression of the endogenous Rubisco enzyme complex) has been reduced or eliminated. In some embodiments, the reduction or elimination of expression comprises use of antisense technology and/or gene editing (e.g., gene knockout). In some embodiments, both Rubisco LSU and SSU are subsequently transformed into the chloroplast genome. Exemplary methods for engineering plants include chloroplast transformation.

In some aspects, the disclosure features a genetically engineered plant comprising a Rubisco LSU comprising any one of the sets of amino acid substitution mutations listed in Table 1, wherein the amino acid substitution mutations are numbered relative to the LSU of Nicotiana tabacum (SEQ ID NO: 43). In some embodiments, the Rubisco LSU comprises an amino acid sequence having at least 80%, 85%, 90%, 95%, 98%, or 99% sequence identity to any one of SEQ ID NOs: 1-19.

In some aspects, the disclosure features a genetically engineered plant comprising a Rubisco SSU comprising any one of the sets of amino acid substitution mutations listed in Table 1, wherein the amino acid substitution mutations are numbered relative to the S-T2 subunit of Nicotiana tabacum (SEQ ID NO: 44). In some embodiments, the Rubisco LSU comprises an amino acid sequence having at least 80%, 85%, 90%, 95%, 98%, or 99% sequence identity to any one of SEQ ID NOs: 20-42.

In another aspect, the disclosure features a genetically engineered plant comprising (a) a Rubisco large subunit (LSU) comprising L225I and K429Q amino acid substitution mutations, wherein the amino acid substitution mutations are numbered relative to the LSU of Nicotiana tabacum (SEQ ID NO: 43); and (b) a Rubisco small subunit (SSU) comprising N8G, V30I, and E88Q amino acid substitution mutations, wherein the amino acid substitution mutations are numbered relative to the S-T2 subunit of Nicotiana tabacum (SEQ ID NO: 44). In some embodiments, the Rubisco LSU comprises an amino acid sequence having at least 80%, 85%, 90%, 95%, 98%, or 99% sequence identity to SEQ ID NO: 1. In some embodiments, the Rubisco LSU comprises the amino acid sequence of SEQ ID NO: 1. In some embodiments, the Rubisco SSU comprises an amino acid sequence having at least 80%, 85%, 90%, 95%, 98%, or 99% sequence identity to SEQ ID NO: 20. In some embodiments, the Rubisco SSU comprises the amino acid sequence of SEQ ID NO: 20. In some aspects, the Rubisco LSU and SSU are Nico1 and Nico1, respectively, as presented in Table 3.

In another aspect, the disclosure features a genetically engineered plant comprising (a) a Rubisco LSU comprising V145I, L225I, and K429Q amino acid substitution mutations, wherein the amino acid substitution mutations are numbered relative to the LSU of Nicotiana tabacum (SEQ ID NO: 43); and (b) a Rubisco SSU comprising N8G, V30I, and E88Q amino acid substitution mutations, wherein the amino acid substitution mutations are numbered relative to the S-T2 subunit of Nicotiana tabacum (SEQ ID NO: 44). In some embodiments, the Rubisco LSU comprises an amino acid sequence having at least 80%, 85%, 90%, 95%, 98%, or 99% sequence identity to SEQ ID NO: 2. In some embodiments, the Rubisco LSU comprises the amino acid sequence of SEQ ID NO: 1. In some embodiments, the Rubisco SSU comprises an amino acid sequence having at least 80%, 85%, 90%, 95%, 98%, or 99% sequence identity to SEQ ID NO: 20. In some embodiments, the Rubisco SSU comprises the amino acid sequence of SEQ ID NO: 20. In some aspects, the Rubisco LSU and SSU are Nico2 and Nico1, respectively, as presented in Table 3.

In another aspect, the disclosure features a genetically engineered plant comprising (a) a Rubisco LSU comprising L225I and K429Q amino acid substitution mutations, wherein the amino acid substitution mutations are numbered relative to the LSU of Nicotiana tabacum (SEQ ID NO: 43); and (b) a Rubisco SSU comprising N8G, K9M, E23D, R28K, V30I, and E88Q amino acid substitution mutations, wherein the amino acid substitution mutations are numbered relative to the S-T2 subunit of Nicotiana tabacum (SEQ ID NO: 44). In some embodiments, the Rubisco LSU comprises an amino acid sequence having at least 80%, 85%, 90%, 95%, 98%, or 99% sequence identity to SEQ ID NO: 1. In some embodiments, the Rubisco LSU comprises the amino acid sequence of SEQ ID NO: 1. In some embodiments, the Rubisco SSU comprises an amino acid sequence having at least 80%, 85%, 90%, 95%, 98%, or 99% sequence identity to SEQ ID NO: 29. In some embodiments, the Rubisco SSU comprises the amino acid sequence of SEQ ID NO: 29. In some aspects, the Rubisco LSU and SSU are Nico1 and SoNi6, respectively, as presented in Table 3.

In another aspect, the disclosure features a genetically engineered plant comprising (a) a Rubisco LSU comprising V911, V145I, L225I, K429Q, E443D, C449S, V466R, A470E, V472M, and V474T amino acid substitution mutations, wherein the amino acid substitution mutations are numbered relative to the LSU of Nicotiana tabacum (SEQ ID NO: 43); and (b) a Rubisco SSU comprising N8G, K9M, S22T, E23D, R28K, V30I, N36K, N56H, E88Q, and Q96N amino acid substitution mutations, wherein the amino acid substitution mutations are numbered relative to the S-T2 subunit of Nicotiana tabacum (SEQ ID NO: 44). In some embodiments, the Rubisco LSU comprises an amino acid sequence having at least 80%, 85%, 90%, 95%, 98%, or 99% sequence identity to SEQ ID NO: 17. In some embodiments, the Rubisco LSU comprises the amino acid sequence of SEQ ID NO: 17. In some embodiments, the Rubisco SSU comprises an amino acid sequence having at least 80%, 85%, 90%, 95%, 98%, or 99% sequence identity to SEQ ID NO: 39. In some embodiments, the Rubisco SSU comprises the amino acid sequence of SEQ ID NO: 39. In some aspects, the Rubisco LSU and SSU are Sofa1 and SoCe1, respectively, as presented in Table 3.

In another aspect, the disclosure features a genetically engineered plant comprising (a) a Rubisco LSU comprising L225I and K429Q amino acid substitution mutations, wherein the amino acid substitution mutations are numbered relative to the LSU of Nicotiana tabacum (SEQ ID NO: 43); and (b) a Rubisco SSU comprising N8G, K9M, E23D, R28K, V30I, K57R, and E88Q amino acid substitution mutations, wherein the amino acid substitution mutations are numbered relative to the S-T2 subunit of Nicotiana tabacum (SEQ ID NO: 44). In some embodiments, the Rubisco LSU comprises an amino acid sequence having at least 80%, 85%, 90%, 95%, 98%, or 99% sequence identity to SEQ ID NO: 1. In some embodiments, the Rubisco LSU comprises the amino acid sequence of SEQ ID NO: 1. In some embodiments, the Rubisco SSU comprises an amino acid sequence having at least 80%, 85%, 90%, 95%, 98%, or 99% sequence identity to SEQ ID NO: 34. In some embodiments, the Rubisco SSU comprises the amino acid sequence of SEQ ID NO: 34. In some aspects, the Rubisco LSU and SSU are Sola2 and Sola3, respectively, as presented in Table 3.

In another aspect, the disclosure features a genetically engineered plant comprising (a) a Rubisco LSU comprising an L225I amino acid substitution mutation, wherein the amino acid substitution mutation is numbered relative to the LSU of Nicotiana tabacum (SEQ ID NO: 43); and (b) a Rubisco SSU comprising K9M, E23D, R28K, V30I, K57R, and E88Q amino acid substitution mutations, wherein the amino acid substitution mutations are numbered relative to the S-T2 subunit of Nicotiana tabacum (SEQ ID NO: 44). In some embodiments, the Rubisco LSU comprises an amino acid sequence having at least 80%, 85%, 90%, 95%, 98%, or 99% sequence identity to SEQ ID NO: 4. In some embodiments, the Rubisco LSU comprises the amino acid sequence of SEQ ID NO: 4. In some embodiments, the Rubisco SSU comprises an amino acid sequence having at least 80%, 85%, 90%, 95%, 98%, or 99% sequence identity to SEQ ID NO: 35. In some embodiments, the Rubisco SSU comprises the amino acid sequence of SEQ ID NO: 35. In some aspects, the Rubisco LSU and SSU are Sola1 and SoJa1, respectively, as presented in Table 3.

In another aspect, the disclosure features a genetically engineered plant comprising (a) a Rubisco LSU comprising L225I and K429Q amino acid substitution mutations, wherein the amino acid substitution mutations are numbered relative to the LSU of Nicotiana tabacum (SEQ ID NO: 43); and (b) a Rubisco SSU comprising K9M, E23D, R28K, V30I, K57R, and E88Q amino acid substitution mutations, wherein the amino acid substitution mutations are numbered relative to the S-T2 subunit of Nicotiana tabacum (SEQ ID NO: 44). In some embodiments, the genetically engineered plant of claim 41, wherein the Rubisco LSU comprises an amino acid sequence having at least 80%, 85%, 90%, 95%, 98%, or 99% sequence identity to SEQ ID NO: 1. In some embodiments, the Rubisco LSU comprises the amino acid sequence of SEQ ID NO: 1. In some embodiments, the Rubisco SSU comprises an amino acid sequence having at least 80%, 85%, 90%, 95%, 98%, or 99% sequence identity to SEQ ID NO: 35. In some embodiments, the Rubisco SSU comprises the amino acid sequence of SEQ ID NO: 35. In some aspects, the Rubisco LSU and SSU are Sola2 and SoJa1, respectively, as presented in Table 3.

In some embodiments of any of the above aspects, the plant that had been modified (e.g., genetically engineered) to comprise the Rubisco LSU and/or Rubisco SSU is a C3 plant. Any C3 plant grown as a crop or horticultural species may be used in the invention. C3 plants that may be used in the invention include, but are not limited to C3 plants in the families Solanaceae, Poaceae, Fabaceae, Brassicaceae, Rosaceae, Euphorbiaceae, Amaranthaceae, and Malvaceae. In some embodiments, the C3 plant is tobacco, tomato, potato, pepper, rice, wheat, barley, soybean, cowpea, peanut, cassava, spinach, or cotton.

In some embodiments, the catalytic efficiency of the Rubisco enzyme complex is increased relative to that of a control Rubisco enzyme complex (e.g., the wild-type Rubisco enzyme complex of tobacco), e.g., increased by at least 1%, 5%, 10%, 15%, 20%, 25%, 30%, 35%, 40%, 45%, 50%, 55%, 60%, 65%, 70%, 75%, 80%, 85%, 90%, 95%, or 100% relative to a control Rubisco enzyme complex.

In some embodiments, the catalytic efficiency of Rubisco in the genetically engineered plant is increased relative to that of a plant not comprising the Rubisco LSU of (a) and the Rubisco SSU of (b) (e.g., relative to a plant comprising a wild-type Rubisco enzyme complex), e.g., increased by at least 1%, 5%, 10%, 15%, 20%, 25%, 30%, 35%, 40%, 45%, 50%, 55%, 60%, 65%, 70%, 75%, 80%, 85%, 90%, 95%, or 100% relative to a plant not comprising the Rubisco LSU of (a) and the Rubisco SSU of (b).

In some embodiments, the k_{cat} value of the Rubisco enzyme complex is increased relative to that of a control Rubisco enzyme complex (e.g., the wild-type Rubisco enzyme complex of tobacco), e.g., increased by at least 1%, 5%, 10%, 15%, 20%, 25%, 30%, 35%, 40%, 45%, 50%, 55%, 60%, 65%, 70%, 75%, 80%, 85%, 90%, 95%, or 100% relative to a control Rubisco enzyme complex.

In some embodiments, the k_{cat} value of Rubisco in the genetically engineered plant is increased relative to that of a plant not comprising the Rubisco LSU of (a) and the Rubisco SSU of (b) (e.g., relative to a plant comprising a wild-type Rubisco enzyme complex), e.g., increased by at least 1%, 5%, 10%, 15%, 20%, 25%, 30%, 35%, 40%, 45%, 50%, 55%, 60%, 65%, 70%, 75%, 80%, 85%, 90%, 95%, or 100% relative to a plant not comprising the Rubisco LSU of (a) and the Rubisco SSU of (b).

In some embodiments, the ribulose-1,5-bisphosphate (RuBP) carboxylation rate of the Rubisco enzyme complex is increased relative to that of a control Rubisco enzyme complex (e.g., the wild-type Rubisco enzyme complex of tobacco), e.g., is increased by 1.1-fold, 1.2-fold, 1.3-fold, 1.4-fold, 1.5-fold, 1.6-fold, 1.7-fold, 1.8-fold, 1.9-fold, 2-fold, or more than 2-fold relative to a control Rubisco enzyme complex.

In some embodiments, the RuBP carboxylation rate of the genetically engineered plant is increased relative to that of a plant not comprising the Rubisco LSU of (a) and the Rubisco SSU of (b) (e.g., relative to a plant comprising a wild-type Rubisco enzyme complex), e.g., is increased by 1.1-fold, 1.2-fold, 1.3-fold, 1.4-fold, 1.5-fold, 1.6-fold, 1.7-fold, 1.8-fold, 1.9-fold, 2-fold, or more than 2-fold relative to a plant not comprising the Rubisco LSU of (a) and the Rubisco SSU of (b).

(i) Wild-Type Nicotiana tabacum (Tobacco) Rubisco Reference Sequences

The wild-type sequence of the Rubisco large subunit (LSU) of Nicotiana tabacum (tobacco) is shown in SEQ ID NO: 43. The wild-type sequence of the Rubisco large subunit (LSU) of Nicotiana tabacum (tobacco) is shown in SEQ ID NO: 43. The wild-type sequence of the Rubisco S-T2 small subunit (SSU) of Nicotiana tabacum (tobacco) is shown in SEQ ID NO: 44.

Wild-type Nicotiana tabacum Rubisco LSU
SEQ ID NO: 43
MSPQTETKASVGFKAGVKEYKLTYYTPEYQTKDTDILAAFRVTPQPGVPP
EEAGAAVAAESSTGTWTTVWTDGLTSLDRYKGRCYRIERVVGEKDQYIAY
VAYPLDLFEEGSVTNMFTSIVGNVFGFKALRALRLEDLRIPPAYVKTFQG
PPHGIQVERDKLNKYGRPLLGCTIKPKLGLSAKNYGRAVYECLRGGLDFT
KDDENVNSQPFMRWRDRFLFCAEALYKAQAETGEIKGHYLNATAGTCEEM
IKRAVFARELGVPIVMHDYLTGGFTANTSLAHYCRDNGLLLHIHRAMHAV
IDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVL
QFGGGTLGHPWGNAPGAVANRVALEACVKARNEGRDLAQEGNEIIREACK
WSPELAAACEVWKEIVFNFAAVDVLDK
Wild-type Nicotiana tabacum Rubisco S-T2 SSU
SEQ ID NO: 44
MQVWPPINKKKYETLSYLPDLSEEQLLREVEYLLKNGWVPCLEFETEHGF
VYRENNKSPGYYDGRYWTMWKLPMFGCTDATQVLAEVEEAKKAYPQAWIR
IIGFDNVRQVQCISFIAYKPEGY

### (ii) Predicted Ancestral Rubisco Sequences

The sequences of predicted ancestral Rubisco LSUs are presented in SEQ ID NOs: 1-19. The sequences of predicted ancestral Rubisco S-T2 SSUs are presented in SEQ ID NOs: 20-42. For each sequence, the header line provided below indicates the sequence name (see Table 3) and the amino acid residue substitutions that differentiate the engineered (ancestral) Rubisco sequence from the appropriate tobacco reference sequence (SEQ ID NO: 43 or SEQ ID NO: 44).

### Ancestral Rubisco Large Subunit Sequences

>Nico1 L225I K429Q (same as Sola2)
SEQ ID NO: 1
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYVKTFQGPPHGIQVERDKLNKYGRPLLGCTIKPKLGLSAKNYGRAVYECLRGGLDFTKDDENVNSQ
PFMRWRDRFLFCAEAIYKAQAETGEIKGHYLNATAGTCEEMIKRAVFARELGVPIVMHDYLTGGFTANTS
LAHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVQARNEGRDLAQEGNEIIREACKWSPELAAACEVWKEIVFNFAAVDVLDK
>Nico2 V145I L225I K429Q
SEQ ID NO: 2
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEAIYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVQARNEGRDLAQEGNEIIREACKWSPELAAACEVWKEIVFNFAAVDVLDK
>Nico3 K429Q
SEQ ID NO: 3
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYVKTFQGPPHGIQVERDKLNKYGRPLLGCTIKPKLGLSAKNYGRAVYECLRGGLDFTKDDENVNSQ
PFMRWRDRFLFCAEALYKAQAETGEIKGHYLNATAGTCEEMIKRAVFARELGVPIVMHDYLTGGFTANTS
LAHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVQARNEGRDLAQEGNEIIREACKWSPELAAACEVWKEIVFNFAAVDVLDK
>Sola1 L225I
SEQ ID NO: 4
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYVKTFQGPPHGIQVERDKLNKYGRPLLGCTIKPKLGLSAKNYGRAVYECLRGGLDFTKDDENVNSQ
PFMRWRDRFLFCAEAIYKAQAETGEIKGHYLNATAGTCEEMIKRAVFARELGVPIVMHDYLTGGFTANTS
LAHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNEIIREACKWSPELAAACEVWKEIVFNFAAVDVLDK
>SoDa1 Y226F
SEQ ID NO: 5
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYVKTFQGPPHGIQVERDKLNKYGRPLLGCTIKPKLGLSAKNYGRAVYECLRGGLDFTKDDENVNSQ
PFMRWRDRFLFCAEALFKAQAETGEIKGHYLNATAGTCEEMIKRAVFARELGVPIVMHDYLTGGFTANTS
LAHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNEIIREACKWSPELAAACEVWKEIVFNFAAVDVLDK
>SoDa2 Y226F S279T Q439R
SEQ ID NO: 6
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYVKTFQGPPHGIQVERDKLNKYGRPLLGCTIKPKLGLSAKNYGRAVYECLRGGLDFTKDDENVNSQ
PFMRWRDRFLFCAEALFKAQAETGEIKGHYLNATAGTCEEMIKRAVFARELGVPIVMHDYLTGGFTANTT
LAHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAREGNEIIREACKWSPELAAACEVWKEIVFNFAAVDVLDK
>SoDa3 (no mutation)
SEQ ID NO: 7
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYVKTFQGPPHGIQVERDKLNKYGRPLLGCTIKPKLGLSAKNYGRAVYECLRGGLDFTKDDENVNSQ
PFMRWRDRFLFCAEALYKAQAETGEIKGHYLNATAGTCEEMIKRAVFARELGVPIVMHDYLTGGFTANTS
LAHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNEIIREACKWSPELAAACEVWKEIVFNFAAVDVLDK
>SoDa4 Y226F S279T
SEQ ID NO: 8
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYVKTFQGPPHGIQVERDKLNKYGRPLLGCTIKPKLGLSAKNYGRAVYECLRGGLDFTKDDENVNSQ
PFMRWRDRFLFCAEALFKAQAETGEIKGHYLNATAGTCEEMIKRAVFARELGVPIVMHDYLTGGFTANTT
LAHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNEIIREACKWSPELAAACEVWKEIVFNFAAVDVLDK
>CaWi1 V145I
SEQ ID NO: 9
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEALYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNEIIREACKWSPELAAACEVWKEIVFNFAAVDVLDK
>CaWi2 V145I S279T
SEQ ID NO: 10
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEALYKAQAETGEIKGHYLNATAGTCEEMIKRAVFARELGVPIVMHDYLTGGFTANTTL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNEIIREACKWSPELAAACEVWKEIVFNFAAVDVLDK
>CaWi3 V145I L219C
SEQ ID NO: 11
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFCFCAEALYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNEIIREACKWSPELAAACEVWKEIVFNFAAVDVLDK
>CaWi4 V145I L219C E443Q
SEQ ID NO: 12
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFCFCAEALYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNQIIREACKWSPELAAACEVWKEIVFNFAAVDVLDK
>CaWi5 V145I S279T Q439R C449S
SEQ ID NO: 13
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEALYKAQAETGEIKGHYLNATAGTCEEMIKRAVFARELGVPIVMHDYLTGGFTANTTL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAREGNEIIREASKWSPELAAACEVWKEIVFNFAAVDVLDK
>CaWi6 V145I L219C E443Q C449S
SEQ ID NO: 14
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFCFCAEALYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVKARNEGRDLAQEGNQIIREASKWSPELAAACEVWKEIVFNFAAVDVLDK
>SoCe1 V145I L225I K429Q C449S V466R A470E V472M V474T
SEQ ID NO: 15
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEAIYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVQARNEGRDLAQEGNEIIREASKWSPELAAACEVWKEIRFNFEAMDTLDK
>SoCe2 V145I L225I K429Q E443D C449S V466R A470E V472M V474T
SEQ ID NO: 16
MSPQTETKASVGFKAGVKEYKLTYYTPEYQTKDTDILAAFRVTPQPGVPPEEAGAAVAAESSTGTWTTV
WTDGLTSLDRYKGRCYRIERVVGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEAIYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVQARNEGRDLAQEGNDIIREASKWSPELAAACEVWKEIRFNFEAMDTLDK
>Sofa1 V91I V145I L225I K429Q E443D C449S V466R A470E V472M V474T
SEQ ID NO: 17
MSPQTETKASVGFKAGVKEYKLTYYTPEYQTKDTDILAAFRVTPQPGVPPEEAGAAVAAESSTGTWTTV
WTDGLTSLDRYKGRCYRIERVIGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEAIYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFVEQDRSRGIYFTQDWVSLPGVLPVASGGIHVWHMPALTEIFGDDSVLQFGGGTLGHPWGNAPGAV
ANRVALEACVQARNEGRDLAQEGNDIIREASKWSPELAAACEVWKEIRFNFEAMDTLDK
>Sofa2 V91I V145I L225I K429Q V354I E443D C449S V466R A470E V472M V474T
SEQ ID NO: 18
MSPQTETKASVGFKAGVKEYKLTYYTPEYQTKDTDILAAFRVTPQPGVPPEEAGAAVAAESSTGTWTTV
WTDGLTSLDRYKGRCYRIERVIGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEAIYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFIEQDRSRGIYFTQDWVSLPGVLPVASGGIHVWHMPALTEIFGDDSVLQFGGGTLGHPWGNAPGAVA
NRVALEACVQARNEGRDLAQEGNDIIREASKWSPELAAACEVWKEIRFNFEAMDTLDK
>Sofa3 V91I V145I L225I K429Q V354I E443D C449S V466R A470E V472M
V474T K477GEKK
SEQ ID NO: 19
MSPQTETKASVGFKAGVKEYKLTYYTPEYQTKDTDILAAFRVTPQPGVPPEEAGAAVAAESSTGTWTTV
WTDGLTSLDRYKGRCYRIERVIGEKDQYIAYVAYPLDLFEEGSVTNMFTSIVGNVFGFKALRALRLEDLRI
PPAYIKTFQGPPHGIQVERDKLNKYGRPLLGCTIKPKLGLSAKNYGRAVYECLRGGLDFTKDDENVNSQP
FMRWRDRFLFCAEAIYKAQAETGEIKGHYLNATAGTCEEMIKRAVFARELGVPIVMHDYLTGGFTANTSL
AHYCRDNGLLLHIHRAMHAVIDRQKNHGIHFRVLAKALRMSGGDHIHSGTVVGKLEGERDITLGFVDLLR
DDFIEQDRSRGIYFTQDWVSLPGVLPVASGGIHVWHMPALTEIFGDDSVLQFGGGTLGHPWGNAPGAVA
NRVALEACVQARNEGRDLAQEGNDIIREASKWSPELAAACEVWKEIRFNFEAMDTLDGEKK

### Ancestral Rubisco Small Subunit Sequences

>Nico1 N8G V30I E88Q
SEQ ID NO: 20
MQVWPPIGKKKYETLSYLPDLSEEQLLREIEYLLKNGWVPCLEFETEHGFVYRENNKSPGYYDGRYWTM
WKLPMFGCTDATQVLAEVQEAKKAYPQAWIRIIGFDNVRQVQCISFIAYKPEGY
>Nico2 17Y N8G V30I E88Q
SEQ ID NO: 21
MQVWPPYGKKKYETLSYLPDLSEEQLLREIEYLLKNGWVPCLEFETEHGFVYRENNKSPGYYDGRYWT
MWKLPMFGCTDATQVLAEVQEAKKAYPQAWIRIIGFDNVRQVQCISFIAYKPEGY
>Nico3 17Y N8G V301 E88G
SEQ ID NO: 22
MQVWPPYGKKKYETLSYLPDLSEEQLLREIEYLLKNGWVPCLEFETEHGFVYRENNKSPGYYDGRYWT
MWKLPMFGCTDATQVLAEVGEAKKAYPQAWIRIIGFDNVRQVQCISFIAYKPEGY
>Nico4 I7Y N8G V30I N55H E88G
SEQ ID NO: 23
MQVWPPYGKKKYETLSYLPDLSEEQLLREIEYLLKNGWVPCLEFETEHGFVYREHNKSPGYYDGRYWT
MWKLPMFGCTDATQVLAEVGEAKKAYPQAWIRIIGFDNVRQVQCISFIAYKPEGY
>SoNi1 K9M V30I E88G
SEQ ID NO: 24
MQVWPPINMKKYETLSYLPDLSEEQLLREIEYLLKNGWVPCLEFETEHGFVYRENNKSPGYYDGRYWTM
WKLPMFGCTDATQVLAEVGEAKKAYPQAWIRIIGFDNVRQVQCISFIAYKPEGY
>SoNi2 K9M E23D R28K V30I E88G (same as Lycium_barbarum_RBCS1)
SEQ ID NO: 25
MQVWPPINMKKYETLSYLPDLSDEQLLKEIEYLLKNGWVPCLEFETEHGFVYRENNKSPGYYDGRYWTM
WKLPMFGCTDATQVLAEVGEAKKAYPQAWIRIIGFDNVRQVQCISFIAYKPEGY
>SoNi3 K9M V30I E88Q
SEQ ID NO: 26
MQVWPPINMKKYETLSYLPDLSEEQLLREIEYLLKNGWVPCLEFETEHGFVYRENNKSPGYYDGRYWTM
WKLPMFGCTDATQVLAEVQEAKKAYPQAWIRIIGFDNVRQVQCISFIAYKPEGY
>SoNi4 N8G K9M V30I E88Q
SEQ ID NO: 27
MQVWPPIGMKKYETLSYLPDLSEEQLLREIEYLLKNGWVPCLEFETEHGFVYRENNKSPGYYDGRYWTM
WKLPMFGCTDATQVLAEVQEAKKAYPQAWIRIIGFDNVRQVQCISFIAYKPEGY
>SoNi5 V30I E88Q
SEQ ID NO: 28
MQVWPPINKKKYETLSYLPDLSEEQLLREIEYLLKNGWVPCLEFETEHGFVYRENNKSPGYYDGRYWTM
WKLPMFGCTDATQVLAEVQEAKKAYPQAWIRIIGFDNVRQVQCISFIAYKPEGY
>SoNi6 N8G K9M E23D R28K V30I E88Q (same as Sola2)
SEQ ID NO: 29
MQVWPPIGMKKYETLSYLPDLSDEQLLKEIEYLLKNGWVPCLEFETEHGFVYRENNKSPGYYDGRYWTM
WKLPMFGCTDATQVLAEVQEAKKAYPQAWIRIIGFDNVRQVQCISFIAYKPEGY
>SoNi7 N8G E23D R28K V30I E88Q
SEQ ID NO: 30
MQVWPPIGKKKYETLSYLPDLSDEQLLKEIEYLLKNGWVPCLEFETEHGFVYRENNKSPGYYDGRYWTM
WKLPMFGCTDATQVLAEVQEAKKAYPQAWIRIIGFDNVRQVQCISFIAYKPEGY
>SoNi8 N8G E23D R28K V30I K57R E88Q
SEQ ID NO: 31
MQVWPPIGKKKYETLSYLPDLSDEQLLKEIEYLLKNGWVPCLEFETEHGFVYRENNRSPGYYDGRYWTM
WKLPMFGCTDATQVLAEVQEAKKAYPQAWIRIIGFDNVRQVQCISFIAYKPEGY
>Sola1 K9M E23D R28K V30I E88Q
SEQ ID NO: 32
MQVWPPINMKKYETLSYLPDLSDEQLLKEIEYLLKNGWVPCLEFETEHGFVYRENNKSPGYYDGRYWTM
WKLPMFGCTDATQVLAEVQEAKKAYPQAWIRIIGFDNVRQVQCISFIAYKPEGY
>Sola2 N8G K9M E23D R28K V30I E88Q (same as SoNi6)
SEQ ID NO: 33
MQVWPPIGMKKYETLSYLPDLSDEQLLKEIEYLLKNGWVPCLEFETEHGFVYRENNKSPGYYDGRYWTM
WKLPMFGCTDATQVLAEVQEAKKAYPQAWIRIIGFDNVRQVQCISFIAYKPEGY
>Sola3 N8G K9M E23D R28K V301 K57R E88Q
SEQ ID NO: 34
MQVWPPIGMKKYETLSYLPDLSDEQLLKEIEYLLKNGWVPCLEFETEHGFVYRENNRSPGYYDGRYWT
MWKLPMFGCTDATQVLAEVQEAKKAYPQAWIRIIGFDNVRQVQCISFIAYKPEGY
>SoJa1 K9M E23D R28K V30I K57R E88Q
SEQ ID NO: 35
MQVWPPINMKKYETLSYLPDLSDEQLLKEIEYLLKNGWVPCLEFETEHGFVYRENNRSPGYYDGRYWTM
WKLPMFGCTDATQVLAEVQEAKKAYPQAWIRIIGFDNVRQVQCISFIAYKPEGY
>CaWi1 K9M E23D R28K V30I K35R A85N E88
QSEQ ID NO: 36
MQVWPPINMKKYETLSYLPDLSDEQLLKEIEYLLRNGWVPCLEFETEHGFVYRENNKSPGYYDGRYWTM
WKLPMFGCTDATQVLNEVQEAKKAYPQAWIRIIGFDNVRQVQCISFIAYKPEGY
>CaWi2 K9M E23D R28K V30I K35R K57R A85N E88Q
SEQ ID NO: 37
MQVWPPINMKKYETLSYLPDLSDEQLLKEIEYLLRNGWVPCLEFETEHGFVYRENNRSPGYYDGRYWT
MWKLPMFGCTDATQVLNEVQEAKKAYPQAWIRIIGFDNVRQVQCISFIAYKPEGY
>CaWi3 K9M E23D R28K V30I K35R N36S K57R A85N E88Q
SEQ ID NO: 38
MQVWPPINMKKYETLSYLPDLSDEQLLKEIEYLLRSGWVPCLEFETEHGFVYRENNRSPGYYDGRYWTM
WKLPMFGCTDATQVLNEVQEAKKAYPQAWIRIIGFDNVRQVQCISFIAYKPEGY
>SoCe1 N8G K9M S22T E23D R28K V30I N36K N56H E88Q Q96N
SEQ ID NO: 39
MQVWPPIGMKKYETLSYLPDLTDEQLLKEIEYLLKKGWVPCLEFETEHGFVYRENHKSPGYYDGRYWTM
WKLPMFGCTDATQVLAEVQEAKKAYPNAWIRIIGFDNVRQVQCISFIAYKPEGY
>SoCe2 N8G S22T E23D R28K V30I N36K N56H E88Q Q96N
SEQ ID NO: 40
MQVWPPIGKKKYETLSYLPDLTDEQLLKEIEYLLKKGWVPCLEFETEHGFVYRENHKSPGYYDGRYWTM
WKLPMFGCTDATQVLAEVQEAKKAYPNAWIRIIGFDNVRQVQCISFIAYKPEGY
>SoCe3 N8G S22T E23D R28K V30I K35N N36K N56H E88Q Q96N
SEQ ID NO: 41
MQVWPPIGKKKYETLSYLPDLTDEQLLKEIEYLLNKGWVPCLEFETEHGFVYRENHKSPGYYDGRYWTM
WKLPMFGCTDATQVLAEVQEAKKAYPNAWIRIIGFDNVRQVQCISFIAYKPEGY
>SoCe4 N8G S22T E23D R28K V30I K35N N36K N56H K57R E88Q Q96N
SEQ ID NO: 42
MQVWPPIGKKKYETLSYLPDLTDEQLLKEIEYLLNKGWVPCLEFETEHGFVYRENHRSPGYYDGRYWTM
WKLPMFGCTDATQVLAEVQEAKKAYPNAWIRIIGFDNVRQVQCISFIAYKPEGY

### Ancestral Rubisco Large Subunit Sequence Alignment

An alignment comparing the amino acid sequences of the nineteen predicted ancestral Rubisco LSUs (SEQ ID NOs: 1-19) is shown below. An asterisk indicates that all of the sequences share the indicated residue at the indicated position. A colon indicates that one or more of the sequences differs at that position.

Rubisco Large Subunit Multiple Sequence Alignment
CLUSTAL O(1.2.4) multiple sequence alignment

| | | |
|---|---|---|
| Sofa3 | MSPQTETKASVGFKAGVKEYKLTYYTPEYQTKDTDILAAFRVTPQPGVPPEEAGAAVAAE | 60 |
| Sofa2 | MSPQTETKASVGFKAGVKEYKLTYYTPEYQTKDTDILAAFRVTPQPGVPPEEAGAAVAAE | 60 |
| Sofa1 | MSPQTETKASVGFKAGVKEYKLTYYTPEYQTKDTDILAAFRVTPQPGVPPEEAGAAVAAE | 60 |
| SoCe1 | MSPQTETKASVGFKAGVKEYKLTYYTPEYQTKDTDILAAFRVTPQPGVPPEEAGAAVAAE | 60 |
| SoCe2 | MSPQTETKASVGFKAGVKEYKLTYYTPEYQTKDTDILAAFRVTPQPGVPPEEAGAAVAAE | 60 |
| CaWi5 | MSPQTETKASVGFKAGVKEYKLTYYTPEYQTKDTDILAAFRVTPQPGVPPEEAGAAVAAE | 60 |
| SoDa2 | MSPQTETKASVGFKAGVKEYKLTYYTPEYQTKDTDILAAFRVTPQPGVPPEEAGAAVAAE | 60 |
| SoDa4 | MSPQTETKASVGFKAGVKEYKLTYYTPEYQTKDTDILAAFRVTPQPGVPPEEAGAAVAAE | 60 |
| Sola1 | MSPQTETKASVGFKAGVKEYKLTYYTPEYQTKDTDILAAFRVTPQPGVPPEEAGAAVAAE | 60 |
| Nico3 | MSPQTETKASVGFKAGVKEYKLTYYTPEYQTKDTDILAAFRVTPQPGVPPEEAGAAVAAE | 60 |
| Nico1 | MSPQTETKASVGFKAGVKEYKLTYYTPEYQTKDTDILAAFRVTPQPGVPPEEAGAAVAAE | 60 |
| Nico2 | MSPQTETKASVGFKAGVKEYKLTYYTPEYQTKDTDILAAFRVTPQPGVPPEEAGAAVAAE | 60 |
| CaWi6 | MSPQTETKASVGFKAGVKEYKLTYYTPEYQTKDTDILAAFRVTPQPGVPPEEAGAAVAAE | 60 |
| CaWi4 | MSPQTETKASVGFKAGVKEYKLTYYTPEYQTKDTDILAAFRVTPQPGVPPEEAGAAVAAE | 60 |
| Cawi3 | MSPQTETKASVGFKAGVKEYKLTYYTPEYQTKDTDILAAFRVTPQPGVPPEEAGAAVAAE | 60 |
| CaWi2 | MSPQTETKASVGFKAGVKEYKLTYYTPEYQTKDTDILAAFRVTPQPGVPPEEAGAAVAAE | 60 |
| CaWi1 | MSPQTETKASVGFKAGVKEYKLTYYTPEYQTKDTDILAAFRVTPQPGVPPEEAGAAVAAE | 60 |
| SoDa1 | MSPQTETKASVGFKAGVKEYKLTYYTPEYQTKDTDILAAFRVTPQPGVPPEEAGAAVAAE | 60 |
| SoDa3 | MSPQTETKASVGFKAGVKEYKLTYYTPEYQTKDTDILAAFRVTPQPGVPPEEAGAAVAAE | 60 |
| | ************************************************************ | |
| Sofa3 | SSTGTWTTVWTDGLTSLDRYKGRCYRIERVIGEKDQYIAYVAYPLDLFEEGSVINMFTSI | 120 |
| Sofa2 | SSTGTWTTVWTDGLTSLDRYKGRCYRIERVIGEKDQYIAYVAYPLDLFEEGSVINMFTSI | 120 |
| Sofa1 | SSTGTWTTVWTDGLISLDRYKGRCYRIERVIGEKDQYIAYVAYPLDLFEEGSVINMFTSI | 120 |
| SoCe1 | SSTGTWTTVWTDGLTSLDRYKGRCYRIERVVGEKDQYIAYVAYPLDLFEEGSVINMFTSI | 120 |
| SoCe2 | SSTGTWTTVWTDGLTSLDRYKGRCYRIERVVGEKDQYIAYVAYPLDLFEEGSVINMFTSI | 120 |
| CaWi5 | SSTGTWTTVWTDGLTSLDRYKGRCYRIERVVGEKDQYIAYVAYPLDLFEEGSVTNMFTSI | 120 |
| SoDa2 | SSTGTWTTVWTDGLTSLDRYKGRCYRIERVVGEKDQYIAYVAYPLDLFEEGSVINMFTSI | 120 |
| SoDa4 | SSTGTWTTVWTDGLTSLDRYKGRCYRIERVVGEKDQYIAYVAYPLDLFEEGSVINMFTSI | 120 |
| Sola1 | SSTGTWTTVWTDGLTSLDRYKGRCYRIERVVGEKDQYIAYVAYPLDLFEEGSVINMFTSI | 120 |
| Nico3 | SSTGTWTTVWTDGLTSLDRYKGRCYRIERVVGEKDQYIAYVAYPLDLFEEGSVINMFTSI | 120 |
| Nico1 | SSTGTWTTVWTDGLTSLDRYKGRCYRIERVVGEKDQYIAYVAYPLDLFEEGSVINMFTSI | 120 |
| Nico2 | SSTGTWTTVWTDGLTSLDRYKGRCYRIERVVGEKDQYIAYVAYPLDLFEEGSVINMFTSI | 120 |
| CaWi6 | SSTGTWTTVWTDGLTSLDRYKGRCYRIERVVGEKDQYIAYVAYPLDLFEEGSVINMFTSI | 120 |
| CaWi4 | SSTGTWTTVWTDGLTSLDRYKGRCYRIERVVGEKDQYIAYVAYPLDLFEEGSVINMFTSI | 120 |
| Cawi3 | SSTGTWTTVWTDGLISLDRYKGRCYRIERVVGEKDQYIAYVAYPLDLFEEGSVINMFTSI | 120 |
| CaWi2 | SSTGTWTTVWTDGLTSLDRYKGRCYRIERVVGEKDQYIAYVAYPLDLFEEGSVINMFTSI | 120 |
| CaWi1 | SSTGTWTTVWTDGLTSLDRYKGRCYRIERVVGEKDQYIAYVAYPLDLFEEGSVINMFTSI | 120 |
| SoDa1 | SSTGTWTTVWTDGLTSLDRYKGRCYRIERVVGEKDQYIAYVAYPLDLFEEGSVINMFTSI | 120 |
| SoDa3 | SSTGTWTTVWTDGLTSLDRYKGRCYRIERVVGEKDQYIAYVAYPLDLFEEGSVINMFTSI | 120 |
| | ************************************************************ | |
| Sofa3 | VGNVFGFKALRALRLEDLRIPPAYIKTFQGPPHGIQVERDKLNKYGRPLLGCTIKPKLGL | 180 |
| Sofa2 | VGNVFGFKALRALRLEDLRIPPAYIKTFQGPPHGIQVERDKLNKYGRPLLGCTIKPKLGL | 180 |
| Sofa1 | VGNVFGFKALRALRLEDLRIPPAYIKTFQGPPHGIQVERDKLNKYGRPLLGCTIKPKLGL | 180 |
| SoCe1 | VGNVFGFKALRALRLEDLRIPPAYIKTFQGPPHGIQVERDKLNKYGRPLLGCTIKPKLGL | 180 |
| SoCe2 | VGNVFGFKALRALRLEDLRIPPAYIKTFQGPPHGIQVERDKLNKYGRPLLGCTIKPKLGL | 180 |
| Cawi5 | VGNVFGFKALRALRLEDLRIPPAYIKTFQGPPHGIQVERDKLNKYGRPLLGCTIKPKLGL | 180 |
| SoDa2 | VGNVFGFKALRALRLEDLRIPPAYVKTFQGPPHGIQVERDKLNKYGRPLLGCTIKPKLGL | 180 |
| SoDa4 | VGNVFGFKALRALRLEDLRIPPAYVKTFQGPPHGIQVERDKLNKYGRPLLGCTIKPKLGL | 180 |
| Sola1 | VGNVFGFKALRALRLEDLRIPPAYVKTFQGPPHGIQVERDKLNKYGRPLLGCTIKPKLGL | 180 |
| Nico3 | VGNVFGFKALRALRLEDLRIPPAYVKTFQGPPHGIQVERDKLNKYGRPLLGCTIKPKLGL | 180 |
| Nico1 | VGNVFGFKALRALRLEDLRIPPAYVKTFQGPPHGIQVERDKLNKYGRPLLGCTIKPKLGL | 180 |
| Nico2 | VGNVFGFKALRALRLEDLRIPPAYIKTFQGPPHGIQVERDKLNKYGRPLLGCTIKPKLGL | 180 |
| CaWi6 | VGNVFGFKALRALRLEDLRIPPAYIKTFQGPPHGIQVERDKLNKYGRPLLGCTIKPKLGL | 180 |
| CaWi4 | VGNVFGFKALRALRLEDLRIPPAYIKTFQGPPHGIQVERDKLNKYGRPLLGCTIKPKLGL | 180 |
| CaWi3 | VGNVFGFKALRALRLEDLRIPPAYIKTFQGPPHGIQVERDKLNKYGRPLLGCTIKPKLGL | 180 |
| Cawi2 | VGNVFGFKALRALRLEDLRIPPAYIKTFQGPPHGIQVERDKLNKYGRPLLGCTIKPKLGL | 180 |
| CaWi1 | VGNVFGFKALRALRLEDLRIPPAYIKTFQGPPHGIQVERDKLNKYGRPLLGCTIKPKLGL | 180 |
| SoDa1 | VGNVFGFKALRALRLEDLRIPPAYVKTFQGPPHGIQVERDKLNKYGRPLLGCTIKPKLGL | 180 |
| SoDa3 | VGNVFGFKALRALRLEDLRIPPAYVKTFQGPPHGIQVERDKLNKYGRPLLGCTIKPKLGL | 180 |
| | ************************************************************ | |
| Sofa3 | SAKNYGRAVYECLRGGLDFTKDDENVNSQPFMRWRDRFLFCAEAIYKAQAETGEIKGHYL | 240 |
| Sofa2 | SAKNYGRAVYECLRGGLDFTKDDENVNSQPFMRWRDRELFCAEAIYKAQAETGEIKGHYL | 240 |
| Sofa1 | SAKNYGRAVYECLRGGLDFTKDDENVNSQPFMRWRDRFLFCAEAIYKAQAETGEIKGHYL | 240 |
| SoCe1 | SAKNYGRAVYECLRGGLDFTKDDENVNSQPFMRWRDRFLFCAEAIYKAQAETGEIKGHYL | 240 |
| SoCe2 | SAKNYGRAVYECLRGGLDFTKDDENVNSQPFMRWRDRFLFCAEAIYKAQAETGEIKGHYL | 240 |
| CaWi5 | SAKNYGRAVYECLRGGLDFTKDDENVNSQPFMRWRDRFLFCAEALYKAQAETGEIKGHYL | 240 |
| SoDa2 | SAKNYGRAVYECLRGGLDFTKDDENVNSQPFMRWRDRFLFCAEALFKAQAETGEIKGHYL | 240 |
| SoDa4 | SAKNYGRAVYECLRGGLDFTKDDENVNSQPFMRWRDRFLFCAEALFKAQAETGEIKGHYL | 240 |
| Sola1 | SAKNYGRAVYECLRGGLDFTKDDENVNSQPFMRWRDRELFCAEAIYKAQAETGEIKGHYL | 240 |
| Nico3 | SAKNYGRAVYECLRGGLDFTKDDENVNSQPFMRWRDRFLFCAEALYKAQAETGEIKGHYL | 240 |
| Nico1 | SAKNYGRAVYECLRGGLDFTKDDENVNSQPFMRWRDRFLFCAEAIYKAQAETGEIKGHYL | 240 |
| Nico2 | SAKNYGRAVYECLRGGLDFTKDDENVNSQPFMRWRDRFLFCAEAIYKAQAETGEIKGHYL | 240 |
| CaWi6 | SAKNYGRAVYECLRGGLDFTKDDENVNSQPFMRWRDRFCFCAEALYKAQAETGEIKGHYL | 240 |
| CaWi4 | SAKNYGRAVYECLRGGLDFTKDDENVNSQPFMRWRDRFCFCAEALYKAQAETGEIKGHYL | 240 |
| Cawi3 | SAKNYGRAVYECLRGGLDFTKDDENVNSQPFMRWRDRFCFCAEALYKAQAETGEIKGHYL | 240 |
| CaWi2 | SAKNYGRAVYECLRGGLDFTKDDENVNSQPFMRWRDRFLFCAEALYKAQAETGEIKGHYL | 240 |
| CaWi1 | SAKNYGRAVYECLRGGLDFTKDDENVNSQPFMRWRDRFLFCAEALYKAQAETGEIKGHYL | 240 |
| SoDa1 | SAKNYGRAVYECLRGGLDFTKDDENVNSQPFMRWRDRFLFCAEALFKAQAETGEIKGHYL | 240 |
| SoDa3 | SAKNYGRAVYECLRGGLDFTKDDENVNSQPFMRWRDRFLFCAEALYKAQAETGEIKGHYL | 240 |
| Sofa3 | NATAGTCEEMIKRAVFARELGVPIVMHDYLTGGFTANTSLAHYCRDNGLLLHIHRAMHAV | 300 |
| Sofa2 | NATAGTCEEMIKRAVFARELGVPIVMHDYLTGGFTANTSLAHYCRDNGLLLHIHRAMHAV | 300 |
| Sofa1 | NATAGTCEEMIKRAVFARELGVPIVMHDYLTGGFTANTSLAHYCRDNGLLLHIHRAMHAV | 300 |
| SoCe1 | NATAGTCEEMIKRAVFARELGVPIVMHDYLTGGFTANTSLAHYCRDNGLLLHIHRAMHAV | 300 |
| SoCe2 | NATAGTCEEMIKRAVFARELGVPIVMHDYLTGGFTANTSLAHYCRDNGLLLHIHRAMHAV | 300 |
| Cawi5 | NATAGTCEEMIKRAVFARELGVPIVMHDYLTGGFTANTTLAHYCRDNGLLLHIHRAMHAV | 300 |
| SoDa2 | NATAGTCEEMIKRAVFARELGVPIVMHDYLTGGFTANTTLAHYCRDNGLLLHIHRAMHAV | 300 |
| SoDa4 | NATAGTCEEMIKRAVFARELGVPIVMHDYLTGGFTANTTLAHYCRDNGLLLHIHRAMHAV | 300 |
| Sola1 | NATAGTCEEMIKRAVFARELGVPIVMHDYLTGGFTANTSLAHYCRDNGLLLHIHRAMHAV | 300 |
| Nico3 | NATAGTCEEMIKRAVFARELGVPIVMHDYLTGGFTANTSLAHYCRDNGLLLHIHRAMHAV | 300 |
| Nico1 | NATAGTCEEMIKRAVFARELGVPIVMHDYLTGGFTANTSLAHYCRDNGLLLHIHRAMHAV | 300 |
| Nico2 | NATAGTCEEMIKRAVFARELGVPIVMHDYLTGGFTANTSLAHYCRDNGLLLHIHRAMHAV | 300 |
| CaWi6 | NATAGTCEEMIKRAVFARELGVPIVMHDYLTGGFTANTSLAHYCRDNGLLLHIHRAMHAV | 300 |
| CaWi4 | NATAGTCEEMIKRAVFARELGVPIVMHDYLTGGFTANTSLAHYCRDNGLLLHIHRAMHAV | 300 |
| Cawi3 | NATAGTCEEMIKRAVFARELGVPIVMHDYLTGGFTANTSLAHYCRDNGLLLHIHRAMHAV | 300 |
| CaWi2 | NATAGTCEEMIKRAVFARELGVPIVMHDYLTGGFTANTTLAHYCRDNGLLLHIHRAMHAV | 300 |
| CaWi1 | NATAGTCEEMIKRAVFARELGVPIVMHDYLTGGFTANTSLAHYCRDNGLLLHIHRAMHAV | 300 |
| SoDa1 | NATAGTCEEMIKRAVFARELGVPIVMHDYLTGGFTANTSLAHYCRDNGLLLHIHRAMHAV | 300 |
| SoDa3 | NATAGTCEEMIKRAVFARELGVPIVMHDYLTGGFTANTSLAHYCRDNGLLLHIHRAMHAV | 300 |
| | ************************************************************ | |
| Sofa3 | IDRQKNHGIHFRVLAKALRMSGGDHIHSGTVVGKLEGERDITLGFVDLLRDDFIEQDRSR | 360 |
| Sofa2 | IDRQKNHGIHFRVLAKALRMSGGDHIHSGTVVGKLEGERDITLGFVDLLRDDFIEQDRSR | 360 |
| Sofa1 | IDRQKNHGIHFRVLAKALRMSGGDHIHSGTVVGKLEGERDITLGFVDLLRDDFVEQDRSR | 360 |
| SoCe1 | IDROKNHGIHFRVLAKALRMSGGDHIHSGTVVGKLEGERDITLGFVDLLRDDFVEQDRSR | 360 |
| SoCe2 | IDRQKNHGIHFRVLAKALRMSGGDHIHSGTVVGKLEGERDITLGFVDLLRDDFVEQDRSR | 360 |
| CaWi5 | IDRQKNHGIHFRVLAKALRMSGGDHIHSGTVVGKLEGERDITLGFVDLLRDDFVEQDRSR | 360 |
| SoDa2 | IDRQKNHGIHFRVLAKALRMSGGDHIHSGTVVGKLEGERDITLGFVDLLRDDFVEQDRSR | 360 |
| SoDa4 | IDRQKNHGIHFRVLAKALRMSGGDHIHSGTVVGKLEGERDITLGFVDLLRDDFVEQDRSR | 360 |
| Sola1 | IDRQKNHGIHFRVLAKALRMSGGDHIHSGTVVGKLEGERDITLGFVDLLRDDEVEQDRSR | 360 |
| Nico3 | IDRQKNHGIHFRVLAKALRMSGGDHIHSGTVVGKLEGERDITLGFVDLLRDDFVEQDRSR | 360 |
| Nico1 | IDRQKNHGIHFRVLAKALRMSGGDHIHSGTVVGKLEGERDITLGFVDLLRDDFVEQDRSR | 360 |
| Nico2 | IDRQKNHGIHFRVLAKALRMSGGDHIHSGTVVGKLEGERDITLGFVDLLRDDFVEQDRSR | 360 |
| CaWi6 | IDROKNHGIHFRVLAKALRMSGGDHIHSGTVVGKLEGERDITLGFVDLLRDDFVEQDRSR | 360 |
| CaWi4 | IDRQKNHGIHFRVLAKALRMSGGDHIHSGTVVGKLEGERDITLGFVDLLRDDEVEQDRSR | 360 |
| CaWi3 | IDRQKNHGIHFRVLAKALRMSGGDHIHSGTVVGKLEGERDITLGFVDLLRDDEVEQDRSR | 360 |
| CaWi2 | IDRQKNHGIHFRVLAKALRMSGGDHIHSGTVVGKLEGERDITLGFVDLLRDDFVEQDRSR | 360 |
| CaWi1 | IDRQKNHGIHFRVLAKALRMSGGDHIHSGTVVGKLEGERDITLGFVDLLRDDFVEQDRSR | 360 |
| SoDa1 | IDRQKNHGIHFRVLAKALRMSGGDHIHSGTVVGKLEGERDITLGFVDLLRDDFVEQDRSR | 360 |
| SoDa3 | IDRQKNHGIHFRVLAKALRMSGGDHIHSGTVVGKLEGERDITLGFVDLLRDDEVEQDRSR | 360 |
| | ************************************************************ | |
| Sofa3 | GIYFTQDWVSLPGVLPVASGGIHVWHMPALTEIFGDDSVLQFGGGTLGHPWGNAPGAVAN | 420 |
| Sofa2 | GIYFTQDWVSLPGVLPVASGGIHVWHMPALTEIFGDDSVLQFGGGTLGHPWGNAPGAVAN | 420 |
| Sofa1 | GIYFTQDWVSLPGVLPVASGGIHVWHMPALTEIFGDDSVLOFGGGTLGHPWGNAPGAVAN | 420 |
| SoCe1 | GIYFTQDWVSLPGVLPVASGGIHVWHMPALTEIFGDDSVLQFGGGTLGHPWGNAPGAVAN | 420 |
| SoCe2 | GIYFTQDWVSLPGVLPVASGGIHVWHMPALTEIFGDDSVLQFGGGTLGHPWGNAPGAVAN | 420 |
| Cawi5 | GIYFTQDWVSLPGVLPVASGGIHVWHMPALTEIFGDDSVLQFGGGTLGHPWGNAPGAVAN | 420 |
| SoDa2 | GIYFTQDWVSLPGVLPVASGGIHVWHMPALTEIFGDDSVLQFGGGTLGHPWGNAPGAVAN | 420 |
| SoDa4 | GIYFTQDWVSLPGVLPVASGGIHVWHMPALTEIFGDDSVLOFGGGTLGHPWGNAPGAVAN | 420 |
| Sola1 | GIYFTQDWVSLPGVLPVASGGIHVWHMPALTEIFGDDSVLOFGGGTLGHPWGNAPGAVAN | 420 |
| Nico3 | GIYFTQDWVSLPGVLPVASGGIHVWHMPALTEIFGDDSVLQFGGGTLGHPWGNAPGAVAN | 420 |
| Nico1 | GIYFTQDWVSLPGVLPVASGGIHVWHMPALTEIFGDDSVLQFGGGTLGHPWGNAPGAVAN | 420 |
| Nico2 | GIYFTQDWVSLPGVLPVASGGIHVWHMPALTEIFGDDSVLQFGGGTLGHPWGNAPGAVAN | 420 |
| CaWi6 | GIYFTQDWVSLPGVLPVASGGIHVWHMPALTEIFGDDSVLOFGGGTLGHPWGNAPGAVAN | 420 |
| CaWi4 | GIYFTQDWVSLPGVLPVASGGIHVWHMPALTEIFGDDSVLOFGGGTLGHPWGNAPGAVAN | 420 |
| CaWi3 | GIYFTQDWVSLPGVLPVASGGIHVWHMPALTEIFGDDSVLQFGGGTLGHPWGNAPGAVAN | 420 |
| CaWi2 | GIYFTQDWVSLPGVLPVASGGIHVWHMPALTEIFGDDSVLQFGGGTLGHPWGNAPGAVAN | 420 |
| CaWi1 | GIYFTQDWVSLPGVLPVASGGIHVWHMPALTEIFGDDSVLQFGGGTLGHPWGNAPGAVAN | 420 |
| SoDa1 | GIYFTQDWVSLPGVLPVASGGIHVWHMPALTEIFGDDSVLOFGGGTLGHPWGNAPGAVAN | 420 |
| SoDa3 | GIYFTQDWVSLPGVLPVASGGIHVWHMPALTEIFGDDSVLQFGGGTLGHPWGNAPGAVAN | 420 |
| | *********************************************************** | |
| Sofa3 | RVALEACVQARNEGRDLAQEGNDIIREASKWSPELAAACEVWKEIRENFEAMDTLDGEKK | 480 |
| Sofa2 | RVALEACVQARNEGRDLAQEGNDIIREASKWSPELAAACEVWKEIRENFEAMDTLDK--- | 477 |
| Sofa1 | RVALEACVQARNEGRDLAQEGNDIIREASKWSPELAAACEVWKEIRFNFEAMDTLDK--- | 477 |
| SoCe1 | RVALEACVQARNEGRDLAQEGNEIIREASKWSPELAAACEVWKEIRFNFEAMDTLDK--- | 477 |
| SoCe2 | RVALEACVQARNEGRDLAQEGNDIIREASKWSPELAAACEVWKEIRENFEAMDTLDK--- | 477 |
| CaWi5 | RVALEACVKARNEGRDLAREGNEIIREASKWSPELAAACEVWKEIVFNFAAVDVLDK--- | 477 |
| SoDa2 | RVALEACVKARNEGRDLAREGNEIIREACKWSPELAAACEVWKEIVENFAAVDVLDK--- | 477 |
| SoDa4 | RVALEACVKARNEGRDLAQEGNEIIREACKWSPELAAACEVWKEIVENFAAVDVLDK--- | 477 |
| Sola1 | RVALEACVKARNEGRDLAQEGNEIIREACKWSPELAAACEVWKEIVENFAAVDVLDK--- | 477 |
| Nico3 | RVALEACVQARNEGRDLAQEGNEIIREACKWSPELAAACEVWKEIVENFAAVDVLDK--- | 477 |
| Nico1 | RVALEACVQARNEGRDLAQEGNEIIREACKWSPELAAACEVWKEIVENFAAVDVLDK--- | 477 |
| Nico2 | RVALEACVQARNEGRDLAQEGNEIIREACKWSPELAAACEVWKEIVENFAAVDVLDK--- | 477 |
| CaWi6 | RVALEACVKARNEGRDLAQEGNQIIREASKWSPELAAACEVWKEIVENFAAVDVLDK--- | 477 |
| CaWi4 | RVALEACVKARNEGRDLAQEGNQIIREACKWSPELAAACEVWKEIVENFAAVDVLDK--- | 477 |
| CaWi3 | RVALEACVKARNEGRDLAQEGNEIIREACKWSPELAAACEVWKEIVENFAAVDVLDK--- | 477 |
| CaWi2 | RVALEACVKARNEGRDLAQEGNEIIREACKWSPELAAACEVWKEIVENFAAVDVLDK--- | 477 |
| CaWi1 | RVALEACVKARNEGRDLAQEGNEIIREACKWSPELAAACEVWKEIVENFAAVDVLDK--- | 477 |
| SoDa1 | RVALEACVKARNEGRDLAQEGNEIIREACKWSPELAAACEVWKEIVENFAAVDVLDK--- | 477 |
| SoDa3 | RVALEACVKARNEGRDLAQEGNEIIREACKWSPELAAACEVWKEIVENFAAVDVLDK--- | 477 |
| | ********;*********;***;*****.**************** *** *;*.** | |

### Ancestral Rubisco Small Subunit Sequence Alignment

An alignment comparing the amino acid sequences of the 23 predicted ancestral Rubisco LSUs (SEQ ID NOs: 20-42) is shown below. An asterisk indicates that all of the sequences share the indicated residue at the indicated position. A colon indicates that one or more of the sequences differs at that position.

Rubisco Small Subunit Multiple Sequence Alignment
CLUSTAL O(1.2.4) multiple sequence alignment

| | | |
|---|---|---|
| SoCe4 | MQVWPPIGKKKYETLSYLPDLTDEQLLKEIEYLLNKGWVPCLEFETEHGFVYRENHRSPG | 60 |
| SoCe3 | MQVWPPIGKKKYETLSYLPDLTDEQLLKEIEYLLNKGWVPCLEFETEHGFVYRENHKSPG | 60 |
| SoCe1 | MQVWPPIGMKKYETLSYLPDLTDEQLLKEIEYLLKKGWVPCLEFETEHGFVYRENHKSPG | 60 |
| SoCe2 | MQVWPPIGKKKYETLSYLPDLTDEQLLKEIEYLLKKGWVPCLEFETEHGFVYRENHKSPG | 60 |
| SoNi1 | MQVWPPINMKKYETLSYLPDLSEEQLLREIEYLLKNGWVPCLEFETEHGFVYRENNKSPG | 60 |
| SoNi3 | MQVWPPINMKKYETLSYLPDLSEEQLLREIEYLLKNGWVPCLEFETEHGFVYRENNKSPG | 60 |
| SoNi5 | MQVWPPINKKKYETLSYLPDLSEEQLLREIEYLLKNGWVPCLEFETEHGFVYRENNKSPG | 60 |
| SoNi4 | MQVWPPIGMKKYETLSYLPDLSEEQLLREIEYLLKNGWVPCLEFETEHGFVYRENNKSPG | 60 |
| Nico4 | MQVWPPYGKKKYETLSYLPDLSEEQLLREIEYLLKNGWVPCLEFETEHGFVYREHNKSPG | 60 |
| Nico3 | MQVWPPYGKKKYETLSYLPDLSEEQLLREIEYLLKNGWVPCLEFETEHGFVYRENNKSPG | 60 |
| Nico1 | MQVWPPIGKKKYETLSYLPDLSEEQLLREIEYLLKNGWVPCLEFETEHGFVYRENNKSPG | 60 |
| Nico2 | MQVWPPYGKKKYETLSYLPDLSEEQLLREIEYLLKNGWVPCLEFETEHGFVYRENNKSPG | 60 |
| CaWi3 | MQVWPPINMKKYETLSYLPDLSDEQLLKEIEYLLRSGWVPCLEFETEHGFVYRENNRSPG | 60 |
| CaWi1 | MQVWPPINMKKYETLSYLPDLSDEQLLKEIEYLLRNGWVPCLEFETEHGFVYRENNKSPG | 60 |
| CaWi2 | MQVWPPINMKKYETLSYLPDLSDEQLLKEIEYLLRNGWVPCLEFETEHGFVYRENNRSPG | 60 |
| Sola3 | MQVWPPIGMKKYETLSYLPDLSDEQLLKEIEYLLKNGWVPCLEFETEHGFVYRENNRSPG | 60 |
| SoNi8 | MQVWPPIGKKKYETLSYLPDLSDEQLLKEIEYLLKNGWVPCLEFETEHGFVYRENNRSPG | 60 |
| SoNi6 | MQVWPPIGMKKYETLSYLPDLSDEQLLKEIEYLLKNGWVPCLEFETEHGFVYRENNKSPG | 60 |
| Sola2 | MQVWPPIGMKKYETLSYLPDLSDEQLLKEIEYLLKNGWVPCLEFETEHGFVYRENNKSPG | 60 |
| SoNi7 | MQVWPPIGKKKYETLSYLPDLSDEQLLKEIEYLLKNGWVPCLEFETEHGFVYRENNKSPG | 60 |
| SoJa1 | MQVWPPINMKKYETLSYLPDLSDEQLLKEIEYLLKNGWVPCLEFETEHGFVYRENNRSPG | 60 |
| SoNi2 | MQVWPPINMKKYETLSYLPDLSDEQLLKEIEYLLKNGWVPCLEFETEHGFVYRENNKSPG | 60 |
| Sola1 | MQVWPPINMKKYETLSYLPDLSDEQLLKEIEYLLKNGWVPCLEFETEHGFVYRENNKSPG | 60 |
| | ****** . ************;;****;******..******************;;;*** | |
| SoCe4 | YYDGRYWTMWKLPMFGCTDATQVLAEVQEAKKAYPNAWIRIIGFDNVRQVQCISFIAYKP | 120 |
| SoCe3 | YYDGRYWTMWKLPMFGCTDATQVLAEVQEAKKAYPNAWIRIIGFDNVRQVQCISFIAYKP | 120 |
| SoCe1 | YYDGRYWTMWKLPMFGCTDATQVLAEVQEAKKAYPNAWIRIIGFDNVRQVQCISFIAYKP | 120 |
| SoCe2 | YYDGRYWTMWKLPMFGCTDATQVLAEVQEAKKAYPNAWIRIIGFDNVRQVQCISFIAYKP | 120 |
| SoNi1 | YYDGRYWTMWKLPMFGCTDATQVLAEVGEAKKAYPQAWIRIIGFDNVRQVQCISFIAYKP | 120 |
| SoNi3 | YYDGRYWTMWKLPMFGCTDATQVLAEVQEAKKAYPQAWIRIIGFDNVRQVQCISFIAYKP | 120 |
| SoNi5 | YYDGRYWTMWKLPMFGCTDATQVLAEVQEAKKAYPQAWIRIIGFDNVRQVQCISFIAYKP | 120 |
| SoNi4 | YYDGRYWTMWKLPMFGCTDATQVLAEVQEAKKAYPQAWIRIIGFDNVRQVQCISFIAYKP | 120 |
| Nico4 | YYDGRYWTMWKLPMFGCTDATQVLAEVGEAKKAYPQAWIRIIGFDNVRQVQCISFIAYKP | 120 |
| Nico3 | YYDGRYWTMWKLPMFGCTDATQVLAEVGEAKKAYPQAWIRIIGFDNVRQVQCISFIAYKP | 120 |
| Nico1 | YYDGRYWTMWKLPMFGCTDATQVLAEVQEAKKAYPQAWIRIIGFDNVRQVQCISFIAYKP | 120 |
| Nico2 | YYDGRYWTMWKLPMFGCTDATQVLAEVQEAKKAYPQAWIRIIGFDNVRQVQCISFIAYKP | 120 |
| CaWi3 | YYDGRYWTMWKLPMFGCTDATQVLNEVQEAKKAYPQAWIRIIGFDNVRQVQCISFIAYKP | 120 |
| CaWi1 | YYDGRYWTMWKLPMFGCTDATQVLNEVQEAKKAYPQAWIRIIGFDNVRQVQCISFIAYKP | 120 |
| CaWi2 | YYDGRYWTMWKLPMFGCTDATQVLNEVQEAKKAYPQAWIRIIGFDNVRQVQCISFIAYKP | 120 |
| Sola3 | YYDGRYWTMWKLPMFGCTDATQVLAEVQEAKKAYPQAWIRIIGFDNVRQVQCISFIAYKP | 120 |
| SoNi8 | YYDGRYWTMWKLPMFGCTDATQVLAEVQEAKKAYPQAWIRIIGFDNVRQVQCISFIAYKP | 120 |
| SoNi6 | YYDGRYWTMWKLPMFGCTDATQVLAEVQEAKKAYPQAWIRIIGFDNVRQVQCISFIAYKP | 120 |
| Sola2 | YYDGRYWTMWKLPMFGCTDATQVLAEVQEAKKAYPQAWIRIIGFDNVRQVQCISFIAYKP | 120 |
| SoNi7 | YYDGRYWTMWKLPMFGCTDATQVLAEVQEAKKAYPQAWIRIIGFDNVRQVQCISFIAYKP | 120 |
| SoJa1 | YYDGRYWTMWKLPMFGCTDATQVLAEVQEAKKAYPQAWIRIIGFDNVRQVQCISFIAYKP | 120 |
| SoNi2 | YYDGRYWTMWKLPMFGCTDATQVLAEVGEAKKAYPQAWIRIIGFDNVRQVQCISFIAYKP | 120 |
| Sola1 | YYDGRYWTMWKLPMFGCTDATQVLAEVQEAKKAYPQAWIRIIGFDNVRQVQCISFIAYKP | 120 |
| | ************************ ** *******;************************ | |
| SoCe4 | EGY | 123 |
| SoCe3 | EGY | 123 |
| SoCe1 | EGY | 123 |
| SoCe2 | EGY | 123 |
| SoNi1 | EGY | 123 |
| SoNi3 | EGY | 123 |
| SoNi5 | EGY | 123 |
| SoNi4 | EGY | 123 |
| Nico4 | EGY | 123 |
| Nico3 | EGY | 123 |
| Nico1 | EGY | 123 |
| Nico2 | EGY | 123 |
| CaWi3 | EGY | 123 |
| CaWi1 | EGY | 123 |
| CaWi2 | EGY | 123 |
| Sola3 | EGY | 123 |
| SoNi8 | EGY | 123 |
| SoNi6 | EGY | 123 |
| Sola2 | EGY | 123 |
| SoNi7 | EGY | 123 |
| SoJa1 | EGY | 123 |
| SoNi2 | EGY | 123 |
| Sola1 | EGY | 123 |
| | *** | |

### II. EXAMPLES

### Example 1. Reversing the Evolution of Rubisco to Prepare Plants for Climate Change

Efficient ancestral Rubiscos from the Solanaceae family have high potential to improve photosynthesis in plants.

### Overview

Plants and photosynthetic organisms possess a remarkably inefficient enzyme named Rubisco that fixes atmospheric CO₂ into organic compounds. Understanding how Rubisco has evolved in response to past climate change is important for attempts to adjust plants to future conditions. The present Example describes development of a computational workflow to assemble de novo both large and small subunits of Rubisco enzymes from transcriptomics data, prediction of sequences for ancestral Rubiscos of the Solanaceae (nightshade) family, and characterization of their kinetics after co-expressing them in Escherichia coli. Predicted ancestors of C₃ Rubiscos were identified that possess superior kinetics and great potential to help plants adapt to anthropogenic climate change. These findings also advance the understanding of the evolution of Rubisco's catalytic traits.

### Introduction

Rubisco (ribulose-1,5-bisphosphate carboxylase/oxygenase; EC 4.1.1.39) catalyzes the first step of the reductive pentose phosphate cycle by fixing CO₂ into ribulose-1,5-bisphosphate (RuBP) (Von Caemmerer, J. Plant Phyisol., 252: 153240, 2020). The catalytic mechanism of Rubisco first arose more than 2.5 billion years ago, prior to the Great Oxidation Event, at a time when there was no need to distinguish CO₂ from oxygen (O₂) (Kacar et al., Geobiology, 15: 628-640, 2017; Shih et al., Nat. Commun., 7: 10382, 2016). As the 02 level rose, evolution resulted in an increase in Rubisco's specificity for CO₂, but the enzyme could no longer eliminate its oxygenase activity, which leads to a counterproductive process called photorespiration and lowers the photosynthetic efficiency (Walker et al., Annu. Rev. Plant Biol., 67: 107-129, 2016). In addition, Rubisco is a slow enzyme with a typical turnover number (k_{cat}) of about 2-5 s⁻¹ in terrestrial plants, necessitating investment of immense plant resources to produce Rubisco in abundance (Bar-On et al., Proc. Natl. Acad. Sci. U.S.A., 116: 4738-4743, 2019). Since Rubisco is a major bottleneck in photosynthesis, understanding how its kinetics evolved in response to changing CO₂ and O₂ levels is crucial to improving its catalysis in crops (Christin et al., Mol. Biol. Evol., 25: 2361-2368, 2008; Kapralov et al., Mol. Biol. Evol., 28: 1491-1503, 2011; Poudel et al., Proc. Natl. Acad. Sci. U.S.A., 117: 30541-30547, 2020; Sharwood et al., Nat. Plants, 2:16186, 2016; Studer et al., Proc. Natl. Acad. Sci. U.S.A., 111: 2223-2228, 2014; Whitney et al., Proc. Nat. Acad. Sci. U.S.A., 108: 14688-14693, 2011).

Form I Rubiscos, found in most oxygenic photosynthetic organisms such as cyanobacteria, algae and plants, are most adapted to aerobic environments and utilize eight small (S) subunits to stabilize four homodimers of large (L) subunits as hexadecameric L₈S₈ complexes (Poudel et al., Proc. Nat. Acad. Sci. U.S.A., 117: 30541-30547, 2020; Banda et al., Nat. Plants, 6: 1158-1166, 2020). In plants and most algae, the L₈S₈ Rubisco is assembled with the L subunit encoded from a single rbcL gene located in the chloroplast genome and the S subunits produced from the RBCS multigene family in the nucleus and imported into the chloroplast. Considerable progress has been made to engineer Rubisco with superior kinetics into plants by modifying either the L subunit (Whitney et al., Proc. Natl. Acad. Sci. U.S.A., 108: 14688-14693, 2011; Lin et al., Plant J., 106: 876-887, 2021; Whitney et al., Proc. Natl. Acad. Sci. U.S.A., 112: 3564-3569, 2015), the S subunit (Donovan et al., Front. Genome Ed., 2: 605614, 2020; Matsumura et al., Mol. Plant, 13: 1570-1581, 2020; Zhang et al., Food Sci. Nutr., 8: 3479-3491, 2020), or both subunits simultaneously (Gunn et al., Proc. Nat. Acad. Sci. U.S.A., 117: 25890-25896, 2020; Martin-Avila et al., Plant Cell, 32: 2898-2916, 2020; Lin et al., Nature, 513: 547-550, 2014). However, the biogenesis of L₈S₈ complexes in the chloroplast stroma of algae and plants is an elaborate process and involves the chaperonins and multiple chaperones (Brutnell et al., Plant Cell, 11: 849-864, 1999; Feiz et al., Plant J., 80: 862-869, 2014; Feiz et al., Plant Cell, 24: 3435-3446, 2012; Vitlin Gruber et al., Trends Plant Sci., 18: 688-694, 2013; Kim et al., Mol. Cells, 35: 402-409, 2013). Consequently, evolutionarily distinct foreign Rubisco subunits are poorly compatible with the host chaperones, leading to either no or insufficient production of functional enzymes (Sharwood et al., Nat. Plants, 2: 16186, 2016; Whitney et al., Proc. Natl. Acad. Sci. U.S.A., 112: 3564-3569, 2015)).

Identifying closely related Rubisco enzymes with superior kinetics is therefore a priority to improve photosynthesis in plants (Galmes et al., Plant Cell Environ., 37: 1989-2001, 2014; Orr et al., Plant Physiol., 172: 707-717, 2016; Prins et al., J. Exp. Bot., 67: 1827-1838, 2016). Biochemical analyses of Rubisco from a wide variety of species indicate that Rubisco enzymes with greatly varying kinetic traits exist in nature (Davidi et al., EMBO J., 39: el 04081, 2020; Flamholz et al., Biochemistry, 58: 3365-3376, 2019; Tcherkez et al., Proc. Natl. Acad. Sci. U.S.A., 103: 7246-7251, 2006; Savir et al., Proc. Natl. Acad. Sci. U.S.A., 107: 3475-3480, 2010). Periodic reductions in atmospheric CO₂ concentrations starting at ˜30 million years (Ma) ago have triggered convergent evolution of a CO₂-concentrating mechanism (CCM) called C₄ photosynthesis in multiple plant families (Christin et al., Curr. Biol., 18: 37-43, 2008). A typical Rubisco in a C₄ plant has a lower affinity for CO₂ and a higher k_{cat} compared to that found in a C₃ plant, which has no CCM (Sharwood et al., Nat. Plants, 2: 16186, 2016; Whitney et al., Proc. Natl. Acad. Sci. U.S.A., 108:14688-14693, 2011; Cummins et al., Front. Plant Sci., 12: 662425, 2021). Because of the rapidly increasing atmospheric CO₂ levels in the past 200 years, the Rubisco enzymes in C₃ plants are likely no longer optimized to the current and future CO₂ levels. Although carbon fixation in C₃ plants would increase at higher CO₂ levels, the increase would be limited by the relatively low k_{cat} of their Rubiscos. Biochemical models predicted that installing selected C₄ Rubiscos in C₃ plants could improve photosynthesis by more than 25% (Sharwood et al., Nat. Plants, 2: 16186, 2016; Zhu et al., Plant Cell Environ., 27: 155-165, 2004). Previous attempts to capture kinetic signatures of C₄ Rubiscos were mostly performed through evolutionary analyses of the L subunits, with limited success (Christin et al., Mol. Biol. Evol., 25: 2361-2368, 2008; Kapralov et al., Mol. Biol. Evol., 28: 1491-1503, 2011; Poudel et al., Proc. Natl. Acad. Sci. U.S.A., 117: 30541-30547, 2020; Studer et al., Proc. Natl. Acad. Sci. U.S.A., 111: 2223-2228, 2014; Bouvier et al., Mol. Biol. Evol., 38: 2880-2896, 2021; Iqbal et al., J. Exp. Bot., 72: 6066-6075, 2021). Despite multiple lines of evidence showing the influence of both subunits on catalysis (Matsumura et al., Mol. Plant, 13: 1570-1581, 2020; Martin-Avila et al., Plant Cell, 32: 2898-2916, 2020; Morita et al., Plant Physiol., 164: 69-79, 2014; Spreitzer et al., Proc. Natl. Acad. Sci. U.S.A., 102:17225-17230, 2005; van Lun et al., J. Am. Chem. Soc., 136: 3165-3171, 2014; Lin et al., Nat. Plants, 6: 1289-1299, 2020), it is still challenging to carry out large-scale phylogenetic analyses of the S subunits in plants due to the lack of available sequences except in a relatively small number of model species.

The present study focuses on deep phylogenetic analyses of both Rubisco subunits to understand the evolution of C₃ Rubiscos in the family Solanaceae. The family Solanaceae was used because any Rubisco modified from a Solanaceous enzyme can be readily expressed in Escherichia coli for characterization of its kinetic properties (Lin et al., Nat. Plants, 6: 1289-1299, 2020; Aigner et al., Science, 358: 1272-1278, 2017) and then introduced into a model Solanaceous plant, Nicotiana tabacum (tobacco), for subsequent investigation of its performance in plants (Martin-Avila et al., Plant Cell, 32: 2898-2916, 2020). A computationally efficient workflow was developed to assemble Rubisco sequences de novo from transcriptomics data generated with next-generation sequencing technologies. Data from the workflow markedly expanded the known sequences of both subunits and allowed prediction of their sequences at multiple ancestral nodes within the Solanaceae from phylogenetic analyses. These predicted ancestral Rubisco enzymes were resurrected using a recently developed Escherichia coli expression system (Lin et al., Nat. Plants, 6: 1289-1299, 2020; Aigner et al., Science, 358: 1272-1278, 2017). Many of these enzymes possess k_{cat} values similar to those from C₄ Rubiscos and exhibit significantly higher catalytic efficiency than C₃ Rubiscos. It is hypothesized that some of these ancestors could predate the emergence of C₄ photosynthesis in several other families and illustrate the evolutionary mechanism of C₃ Rubisco through past climate changes. These ancestral Rubisco enzymes appear to be particularly promising candidates to improve photosynthesis in C₃ plants.

### Results:

### (a) De Novo Assembly of Rubisco Sequences

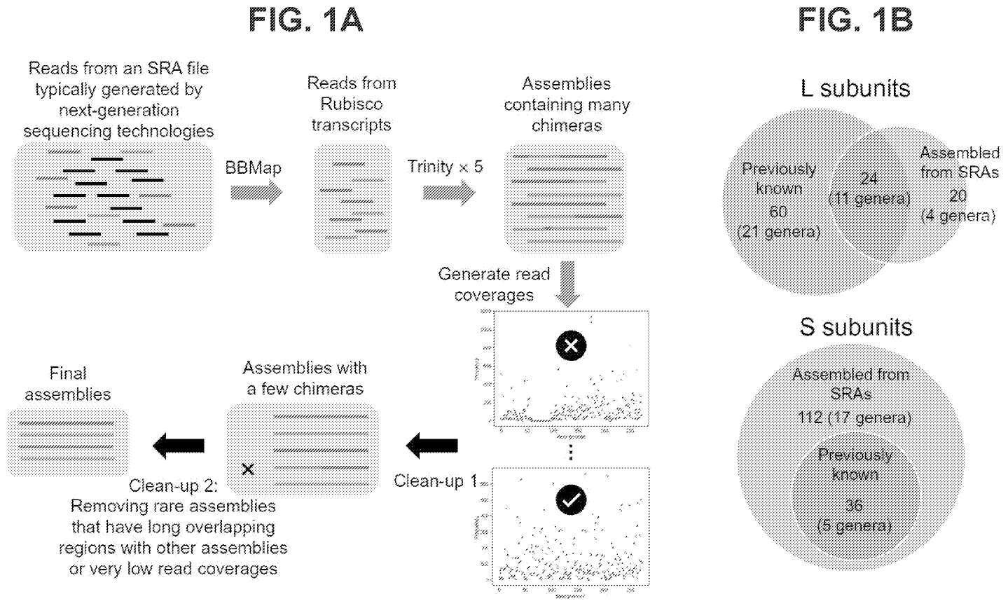

De novo assembly of Rubisco sequences began with Sequence Read Archives (SRAs) containing raw sequences from Solanaceous species at the National Center for Bio-technology Information (NCBI) public repository, which were previously generated with next-generation sequencing. Trinity is one of most frequently used bioinformatic programs for de novo assembly of transcript sequences from SRA files (Grabherr et al., Nat. Biotechnol., 29: 644-652, 2011; Wang and Gribskov, Bioinformatics, 33: 327-333, 2017). A typical SRA file's size is several GBs with millions of reads derived from thousands of transcripts. As a result, using entire SRA files for de novo assembly is computationally intensive. Since the targets include sequences only from the two Rubisco subunits, relevant reads were first extracted using the BBMap program (FIG. 1A). Next, for each set of reads extracted from an SRA, de novo assembly of the Rubisco transcripts was performed with Trinity (Grabherr et al., Nat. Biotechnol., 29: 644-652, 2011) under different configurations. Generation of chimeric sequences with de novo assembly is inevitable especially when multiple paralogs with high sequence homology are present. Thus, the known transcript sequences of the S subunits from several model Solanaceae species, such as tobacco (Nicotiana tabacum), tomato (Solanum lycopersicum) and pepper (Capsicum annuum), were used as benchmarks to evaluate the accuracy of the assemblies. the majority of the assemblies were found to be chimeras due to pervasive overlaps among the rbcS paralogs. Thus, two sequential clean-up steps were implemented to identify and remove potential chimeras: (1) chimeras with overlaps shorter than the read length can be readily recognized from the gaps in their read coverages of starting bases, and (2) chimeras having long overlaps were found to be assembled much less frequently than the authentic transcripts over multiple Trinity runs and were excluded from the final assemblies (FIG. 1A). Assemblies with extremely low read coverages were also removed since they are unlikely to be physiologically important. The workflow was tested with multiple SRAs from each model species and all chimeric sequences were removed reliably although some authentic rbcS transcripts from tobacco were not assembled even after multiple Trinity runs.

Most of the de novo assembly workflow was automated, starting from fetching each SRA file from the online repository up to generating images of read coverages used in the first clean-up step with Python scripts that can be executed in Windows Subsystem for Linux (FIG. 1A). This approach is computationally efficient and can assemble Rubisco sequences from dozens of SRAs a day simply with a modern personal computer equipped with the Windows 10 operating system and high-speed internet. Sequences were assembled from 119 publicly available SRA files to obtain 44 unique L subunits and 134 unique S subunits from 15 Solanaceae genera (FIG. 1B, and Table 1). Remarkably, Trinity was able to assemble complete L subunit sequences from most SRAs even though the data were typically generated from samples enriched with nuclear transcripts. In fact, few chimeras were generated in the assemblies of L subunit sequences, requiring only minimal post-assembly quality control.

TABLE 1
Summary of the Solanaceae Rubisco L and S subunit sequences obtained
with de novo assembly and the numbers of unique protein sequences
after potential chimeras were removed with two clean-up steps.

| | L subunit | | | S subunit | | |
|---|---|---|---|---|---|---|
| | | | Outgroup | | | Outgroups |
| | NCBI | NCBI | (NCBI | NCBI | New | (NCBI |
| | (SRAs) | (proteins) | SRAs) | (SRAs) | (SRAs) | SRAs) |
| Genera | 15 | 21 | 1 | 15 | 7 | 2 |
| Species | 80 | 60 | 1 | 85 | 7 | 4 |
| SRAs | 116 | N/A | 1 | 119 | 17 | 5 |
| Assemblies from Trinity | 554 | N/A | 4 | 3864 | 821 | 54 |
| Final assemblies (Clean-up 1) | 506 | N/A | 4 | 1372 | 104 | 31 |
| Final assemblies (Clean-up 2) | 80 | N/A | 1 | 1299 | 104 | 23 |
| Subunits with duplicates | 80 | N/A | 1 | 206 | 18 | 6 |
| Unique subunits | 44 | 60 | 1 | 134 | 14 | 5 |

Because species belonging to the Solanum and Nicotiana genera were overrepresented in the publicly available sequences, the present study aimed to expand the number of sequences from a more diverse range of genera from the Solanaceae, with a particular focus on those genera that diverged early in the family's evolution such as Fabiana, Browallia, Schizanthus, and Vestia, as well as those that emerged from the common ancestor of Solanum and Nicotiana such as Anthocercis, Nicandra, and Jaborosa. Additional RNA sequencing (RNA-seq) experiments were performed on complementary DNAs (cDNAs) enriched with S subunit sequences using leaf samples from those seven additional genera and added the sequences for 14 S subunits (Table 1).

### (b) Predicting Ancestral Rubisco Sequences

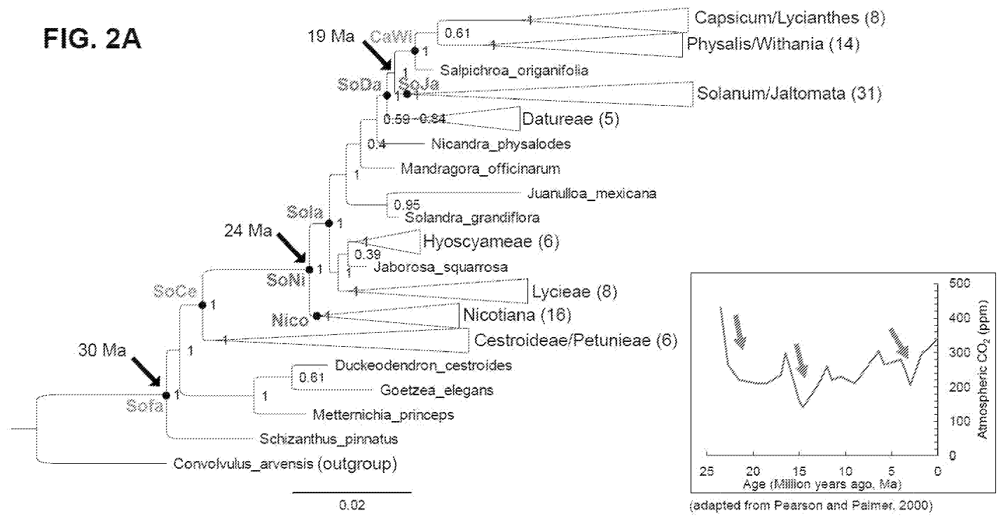

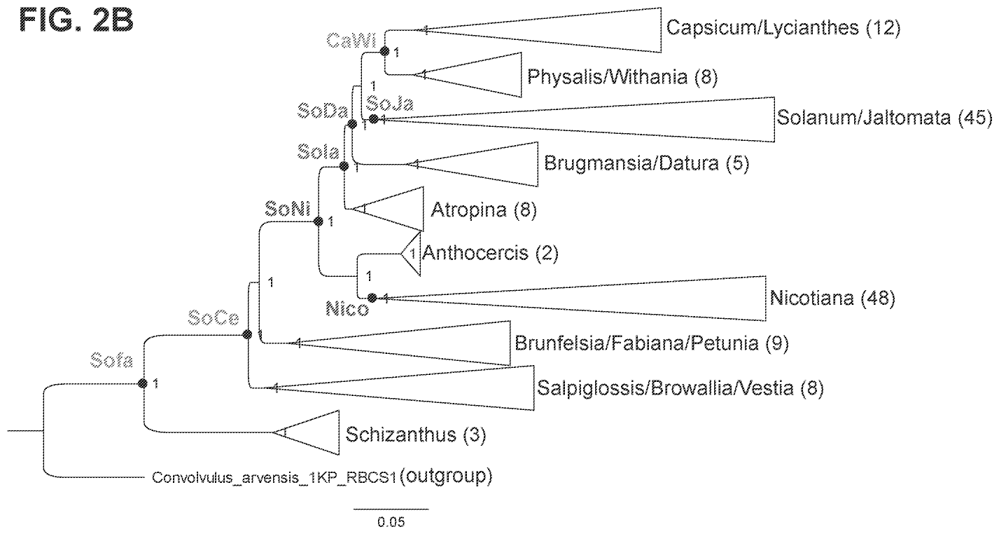

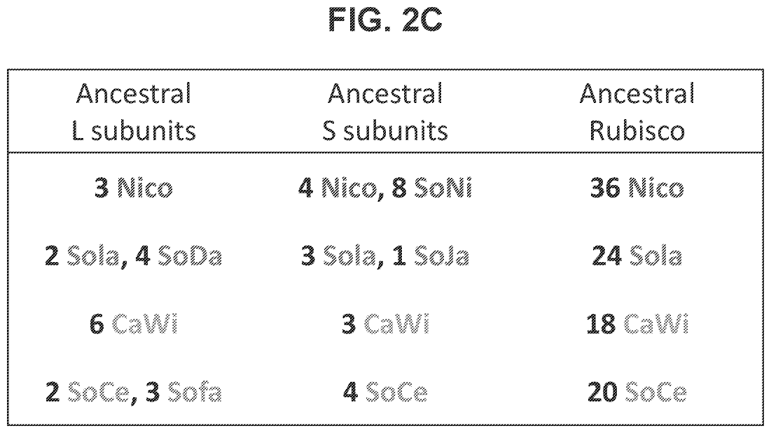

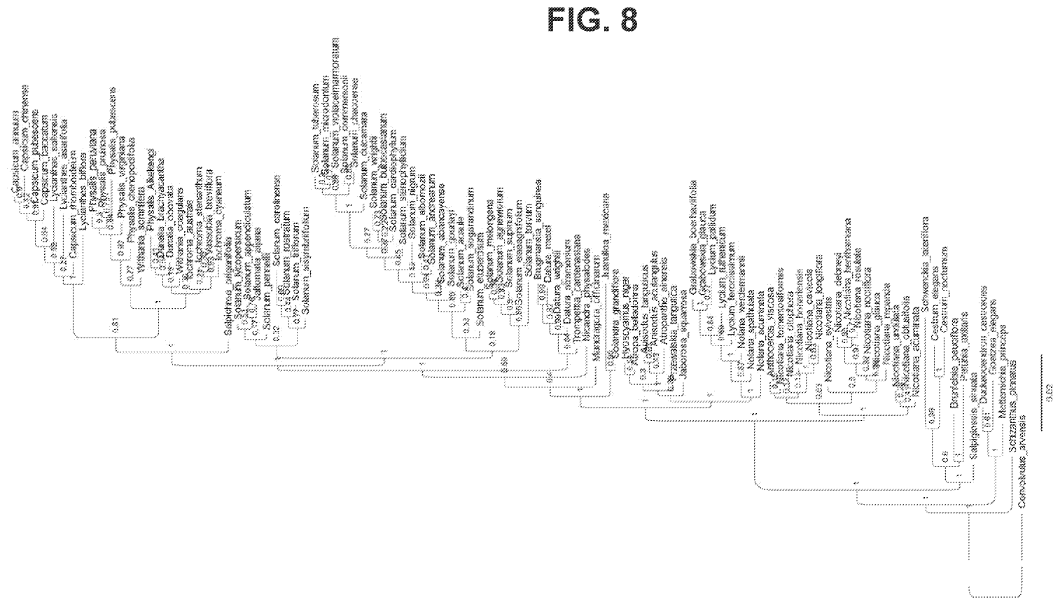

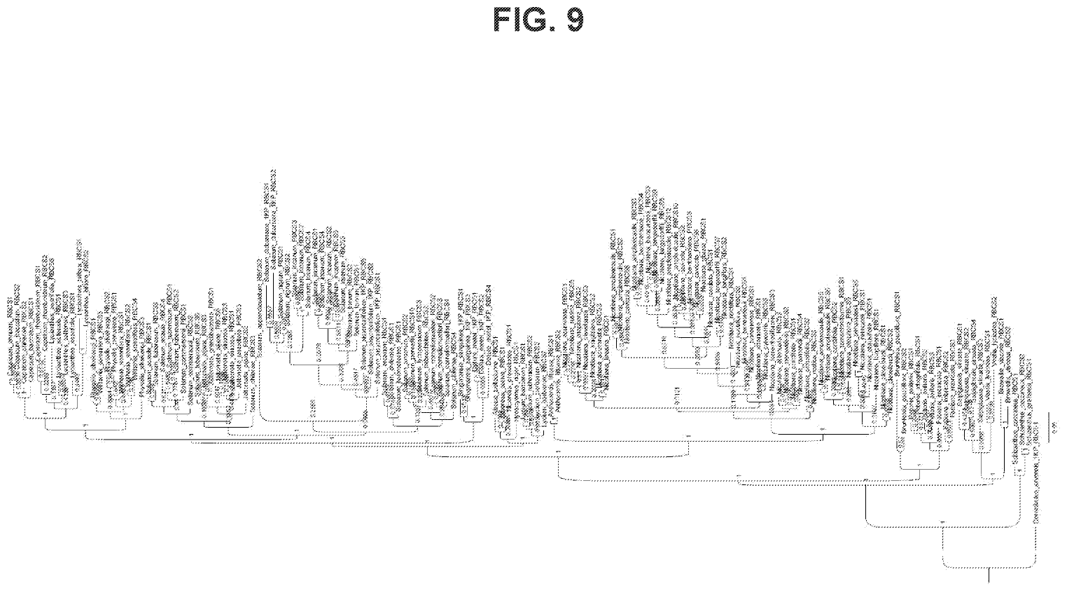

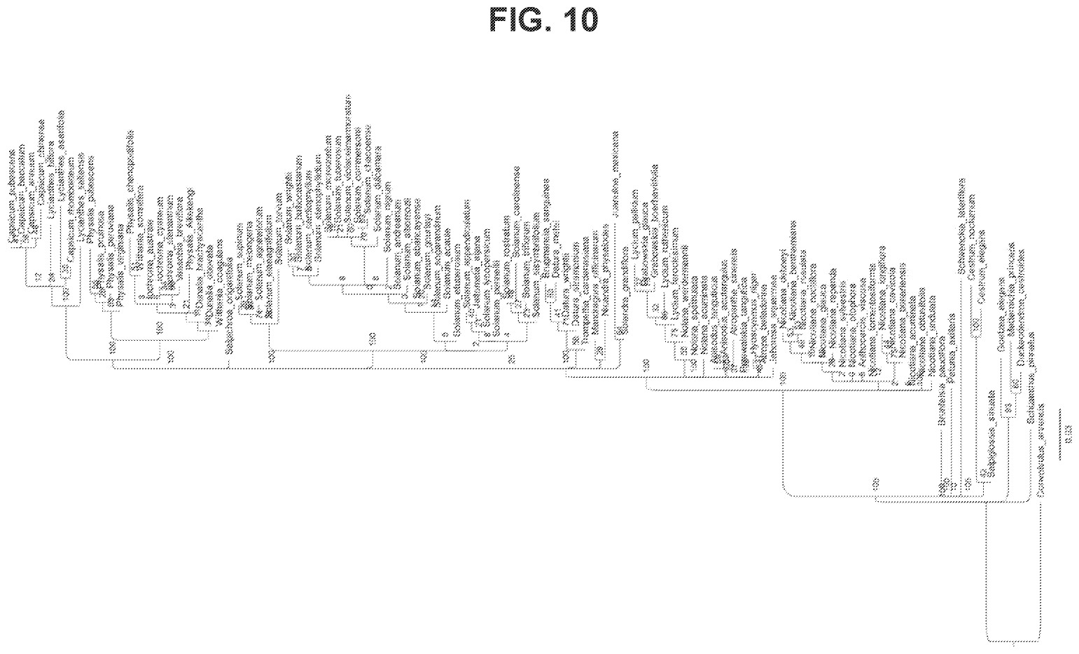

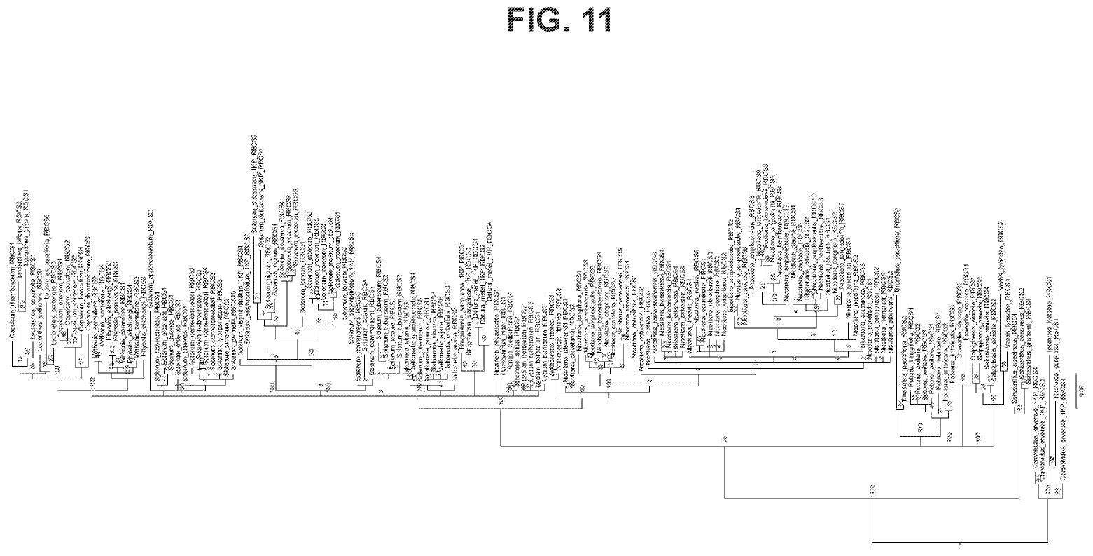

Next, two widely used methods for phylogenetic inference were applied, namely Bayesian inference and maximum likelihood, with the newly expanded protein sequences of L and S subunits from Solanaceae generated both from mining existing sequences and from the additional RNA-seq experiments (FIGS. 2A, 2B, and 8-11). Since the Rubisco subunits are extremely conserved and not suitable for deriving phylogenetic history, constraints were placed at all major nodes that are consistent with the consensus Solanaceae phylogeny (Sarkinen et al., BMC Evol. Biol., 13: 214, 2013). FIG. 2A also displays three nodes within the family with fossil-calibrated divergence time points and the historical CO₂ levels estimated for a similar timeframe showing periodic reductions in the CO₂ levels that presumably gave rise to C₄ photosynthesis in many other families (Sarkinen et al., BMC Evol. Biol., 13: 214, 2013; Pearson et al., Nature, 406: 695-699, 2000). Eight ancestral nodes were named (for example, CaWi for the clade including Capsicum, Lycianthes, Physalis and Withania genera; SoJa for the clade including Solanum and Jaltomata genera) and separated into four colored groups based on the similarity among the predicted residue substitutions (FIGS. 2A and 2B, and Table 2). Both Bayesian inference and maximum likelihood generally produced similar predictions, from which 20 and 23 highly probable L and S subunit sequences, respectively, were derived at these nodes, giving rise to 98 predicted ancestral Rubiscos for further characterization (FIG. 20 and Table 4).

TABLE 2
Predicted residue substitutions in the ancestral subunits compared
to L and S-T2 subunits from tobacco.
Posterior probabilities below 0.80 from Bayesian inference and maximum
likelihood approaches are also included. Those without the
probabilities attached have probabilities above 0.80.

| | L subunit | | S subunit | |
|---|---|---|---|---|
| Ancestral | Bayesian | Maximum | Bayesian | Maximum |
| node | inference | likelihood | inference | likelihood |
| Nico | V145I(.05), | L225I, | I7Y(.14), N8G, V30I, | I7Y(.28), N8G, V30I, |
| | L225I(.69), | K429Q | K57R(.11), | N55H(.30), V87L(.20), |
| | K429Q(.79) | | E88G(.47)/Q(.20) | E88G(.55)/Q(.37) |
| SoNi | V145I(.05), | L225I, | N8G, K9M(.53), E23D, | N8G(.35), K9M, K23D(.48), |
| | L225I(.70), | K429Q | R28K, V30I, K57R(.30), | R28K(.30), V30I, E88Q |
| | K429Q(.78) | | E88Q | |
| SoCe | V91I(.06), | V145I, L225I, | N8G, K9M(.51), S22T, | I7V(.05), N8G, K9M(.18), |
| | V145I, | A228S(.03), | E23D, R28K, V30I, | S22T, E23D, R28K, V30I, |
| | L225I, | K429Q, | K35N(.40), N36K, | K35N, N36K, V39I(.09), |
| | V354I(.06), | E443D(.10), | N56H(.79), | N56H, K57R(.14)/N(.07), |
| | K429Q, | C449S, | K57R(.27)/S(.11), | E88Q, Q96N |
| | E443D(.48), | V466R, | E88Q, | |
| | C449S, | A470E, | Q96N(.73)/S(.25), | |
| | V466R, | V472M, | I99V(.06) | |
| | A470E(.80), | V474T | | |
| | V472M(.65), | | | |
| | V474T | | | |
| Sofa | V91I(.74), | V91I(.31), | N8G, K9M(.42)/L(.09), | N8G(.43), K9M(.61)/L(.15), |
| | V145I(.80), | V145I, | D20P, S22T, E23D, | D20P(.29)/E(.05), S22T, |
| | L225I, | L225I, | L27I, R28K, V30I(.14), | E23D, |
| | I309M(.23), | V354I(.46), | K35N(.37), N36K, | L27I(.44)/M(.06)/V(.05), |
| | S328A(.11), | K429Q, | V39I(.06), N55Y, | R28K, V30I, K35N(.44), |
| | V354I(.56), | E443D, | N56H(.77), | N36K(.49), V39I(.06), |
| | K429Q, | C449S, | K57S(.57)/A(.13)/T(.06)/ | T46L(.27), |
| | E443D(.75), | V466R, | R(.05), E88Q, | N55Y(.50)/H(.05), |
| | C449S, | A470E, | A90V(.47)/C(.07), | N56H(.65), |
| | V466R, | V472M, | Q96S(.51)/N(.32)/G(.12), | K57S(.38)/N(.13)/R(.10)/ |
| | A470E, | V474T | I99V, V107K | T(.05), V87L(.47), E88Q, |
| | V472M(.63), | | | A90V(.40), |
| | V474T, | | | Q96N(.63)/S(.18)/D(.07), |
| | K477R(.24) | | | W98F(.14), I99V(.28), |
| | | | | V107K(.24)/I(.06)/M(.09), |
| | | | | Y118A(.10), E121D(.05) |
| SoIa | L225I(.67), | L225I | K9M, K23D, R28K, | K9M, E23D, R28K, V30I, |
| | K429Q(.12) | | V30I, K57R(.34), E88Q | E88Q |
| SoDa | Y226F, | V145I(.10), | K9M, E23D, R28K, | K9M, E23D, R28K, V30I, |
| | S279T(.49), | Y226F(.15) | V30I, K57R(.33), E88Q | E88Q |
| | Q439R(.23) | | | |
| SoJa | Y226F, | Y226F, | K9M, E23D, R28K, | K9M, E23D, R28K, V30I, |
| | S279T(.75), | S279T, | V30I, E88Q | E88Q |
| | Q439R(.25), | V472M(.06) | | |
| | C449S(.05) | | | |
| CaWi | V145I(.38), | V145I, | K9M, E23D, R28K, | K9M, E23D, R28K, V30I, |
| | S279T(.51), | L219C(.01), | V30I, K35R, N36S(.09), | K35R, N36S(.46)/K(.11), |
| | Q439R(.15), | V354I(.02), | N55H(.06), K57R(.24), | N55H(.11), K57R(.67), |
| | C449S(.06) | E443Q(.03), | A85N(.46), E88Q | A85N, E88Q |
| | | C449S(.32) | | |

Compared to the tobacco subunits, the ancestral Land S subunits have up to 12 and 11 mutations, respectively. Notably, the L sub-units contain fewer changes than the S subunits except for the Sofa and SoCe ancestors. All three Nico L subunits and four of six Sola and SoDa L subunits are identical to extant Solanaceae L subunits, while only 1 of 23 ancestral S subunits, SoNi2, is found in the extant sequences (Table 3). These findings suggest that the evolution of 03 Rubiscos in response to the climate change in the past 30 Ma has been driven more by changes in the S subunits than in the L subunits.

TABLE 3
Summary of residue substitutions in the L and S subunits
of 98 predicted ancestral Rubisco enzymes.

| The identities of extant subunits with | | | | |
|---|---|---|---|---|
| the same sequences are also listed. | | | | |
| Predicted ancestral L subunits | | Predicted ancestral S subunits | | |
| | Residue substitutions | | Residue substitutions | Number of |
| | compared to the L subunit of | | compared to the S-T2 subunit | ancestral |
| Name | tobacco (SEQ ID NO: 43) | Name | of tobacco (SEQ ID NO: 44) | Rubiscos |
| Nico1 | L225I K429Q | Nico1 | N8G V30I E88Q | 36 |
| | (SoIa2 L, Nicotiana | Nico2 | I7Y N8G V30I E88Q | |
| | acuminata L) | Nico3 | I7Y N8G V30I E88G | |
| Nico2 | V145I L225I K429Q | Nico4 | I7Y N8G V30I N55H E88G | |
| | (Nicotiana | SoNi1 | K9M V30I E88G | |
| | undulata L) | SoNi2 | K9M E23D R28K V30I E88G | |
| Nico3 | K429Q (Nicotiana | | (Lycium barbarum RBCS1) | |
| | tomentosiformis L) | SoNi3 | K9M V30I E88Q | |
| SoCe1 | V145I L225I K429Q C449S | SoNi4 | N8G K9M V30I E88Q | |
| | V466R A470E V472M | SoNi5 | V30I E88Q | |
| | V474T | SoNi6 | N8G K9M E23D R28K V30I | |
| SoCe2 | V145I L225I K429Q E443D | | E88Q (SoIa2 S) | |
| | C449S V466R A470E | SoNi7 | N8G E23D R28K V30I E88Q | |
| | V472M V474T | SoNi8 | N8G E23D R28K V30I K57R | |
| Sofa1 | V91I V145I L225I K429Q E443D | | E88Q | |
| | C449S V466R | SoCe1 | N8G K9M S22T E23D R28K | 20 |
| | A470E V472M V474T | | V30I N36K N56H E88Q Q96N | |
| Sofa2 | V91I V145I L225I V354I K429Q | SoCe2 | N8G S22T E23D R28K V30I | |
| | E443D C449S | | N36K N56H E88Q Q96N | |
| | V466R A470E V472M V474T | SoCe3 | N8G S22T E23D R28K V30I | |
| Sofa3 | V91I V145I L225I V354I K429Q | | K35N N36K N56H E88Q Q96N | |
| | E443D C449S V466R A470E | SoCe4 | N8G S22T E23D R28K V30I | |
| | V472M V474T K477GEKK | | K35N N36K N56H K57R E88Q | |
| SoIa1 | L225I (Przewalskia tangutica L) | | Q96N | |
| SoIa2 | L225I K429Q (Nico1 L, N. | SoIa1 | K9M E23D R28K V30I E88Q | 24 |
| | acuminata L) | SoIa2 | N8G K9M E23D R28K V30I | |
| SoDa1 | Y226F | | E88Q (SoNi6 S) | |
| SoDa2 | Y226F S279T Q439R (Solanum | SoIa3 | N8G K9M E23D R28K V30I | |
| | pennellii L) | | K57R E88Q | |
| SoDa3 | None (Atropa belladonna L, | SoJa1 | K9M E23D R28K V30I K57R | |
| | Nicotiana sylvestris L) | | E88Q | |
| SoDa4 | Y226F S279T | CaWi1 | K9M E23D R28K V30I K35R | 18 |
| CaWi1 | V145I (Salpichroa origanifolia L) | | A85N E88Q | |
| CaWi2 | V145I S279T | CaWi2 | K9M E23D R28K V30I K35R | |
| CaWi3 | V145I L219C | | K57R A85N E88Q | |
| CaWi4 | V145I L219C E443Q | CaWi3 | K9M E23D R28K V30I K35R | |
| CaWi5 | V145I S279T Q439R C449S | | N36S K57R A85N E88Q | |
| CaWi6 | V145I L219C E443Q C449S | | | |

### (c) Ancestral Rubiscos are More Efficient

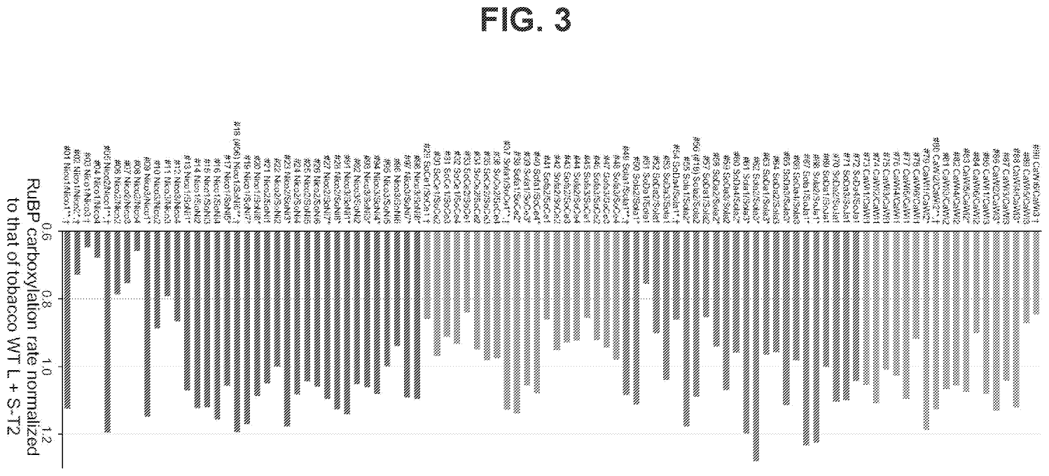

The 98 predicted ancestral Rubisco enzymes of Solanaceae were produced using two expression plasmids that had been previously adapted to produce tobacco Rubisco in E. coli by co-expressing essential chaperonins and chaperones (Lin et al., Nat. Plants, 6: 1289-1299, 2020; Aigner et al., Science, 358: 1272-1278, 2017). The RuBP carboxylation activities of these enzymes were screened at a saturating [CO₂] using their soluble E. coli extracts. None of the residue substitutions led to a total loss of activity, as all samples displayed robust carboxylation activities. Their activities, when normalized with the Rubisco active sites, ranged from about 65% to 128% of the control sample expressing tobacco wild-type (WT) L and S-T2 subunits, with more than half of the predicted ancestors having similar or higher carboxylation rates (FIG. 3). Multiple sequences were tested for both L and S subunits at each node because of the nature of ambiguity associated with predicting ancestral sequences and biases arising from incomplete data, which represented only 36 and 22 genera for L and S subunits, respectively, out of 92 known genera in Solanaceae. As a result, different catalytic rates were observed for the predicted ancestral Rubiscos at each node likely due to differences in either the S subunits or the L subunits. For example, the Nico ancestors with Nico2, Nico3 and Nico4 S subunits displayed markedly lower carboxylation rates than those with Nico1 S subunits regardless of the L subunits. Among the Sola ancestors, those with Sola1 and Sola2 L subunits have consistently higher carboxylation rates than those with SoDa 1 to SoDa4 L subunits (FIG. 3).

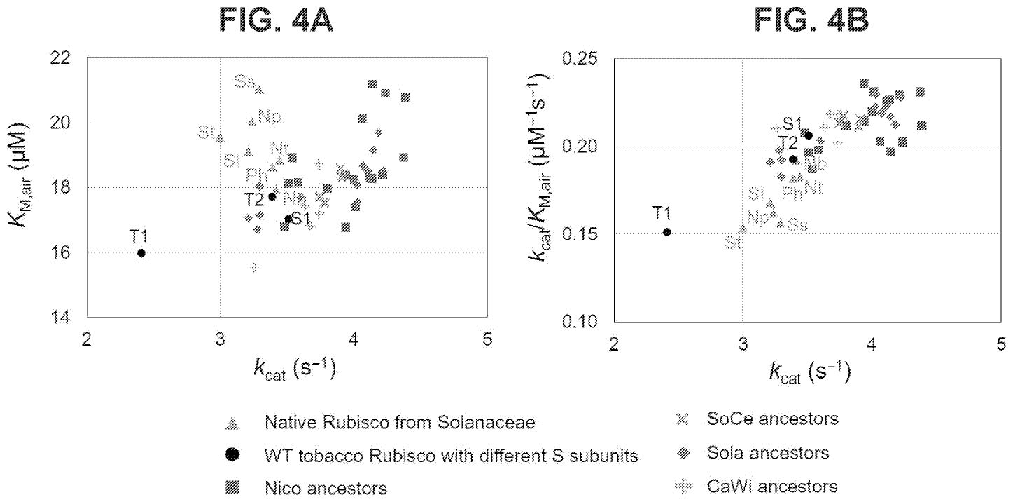

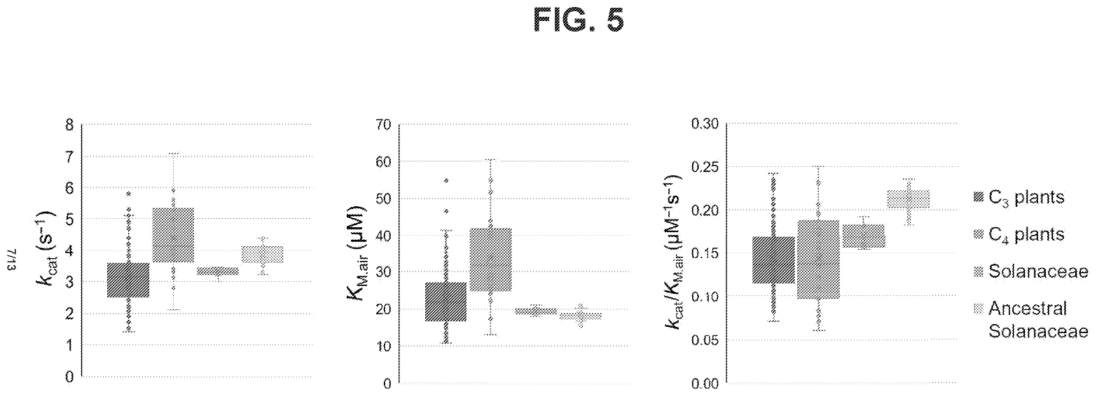

As one of the main goals of the present study was to identify Rubisco enzymes with improved catalysis, 38 predicted ancestors were selected, 34 of which displayed higher RuBP carboxylation activities in the initial screening, for measurement of their RuBP carboxylation rates at six different [CO₂] levels under air at 25° C. along with native Rubisco extracted from leaf tissues of seven Solanaceae species and three E. coli control samples expressing tobacco WT L and either S-S1, S-T1, or S-T2 subunits. The k_{cat} values obtained from these measurements are consistent with their carboxylation activities at the saturating [CO₂] (FIGS. 3 and 4). Several enzymes assembled with Nico L+Nico/SoNi S or Sola L+Sola S subunits have k_{cat} values that are substantially higher than those from the controls and similar to the reported k_{cat} values of typical C₄ Rubiscos (FIG. 5; Sharwood et al., Nat. Plants, 2: 16186, 2016; Whitney et al., Proc. Natl. Acad. Sci. U.S.A., 108: 14688-14693, 2011). All of the ancestral enzymes displayed a similar range of Michaelis constants for CO₂ (K_{M,air}) as the control samples. Notably, there appears to be positive correlation between the catalytic efficiencies (k_{cat}/K_{M,air}) and k_{cat} with many of the ancestors with high k_{cat} also having elevated catalytic efficiencies (FIGS. 4B and 5). Several of those predicted ancestors with extant L subunits such as Nico1, Nico2, Sola1, and Sola2 L subunits (#1, #5, #18, #19, #23, #61, #62, and #67) display higher k_{cat} and carboxylation efficiency than the extant Solanaceae and C₃ Rubiscos in general (Table 3 and FIG. 5). Hence, S subunits likely play crucial roles in improving the kinetics of these ancestral enzymes.

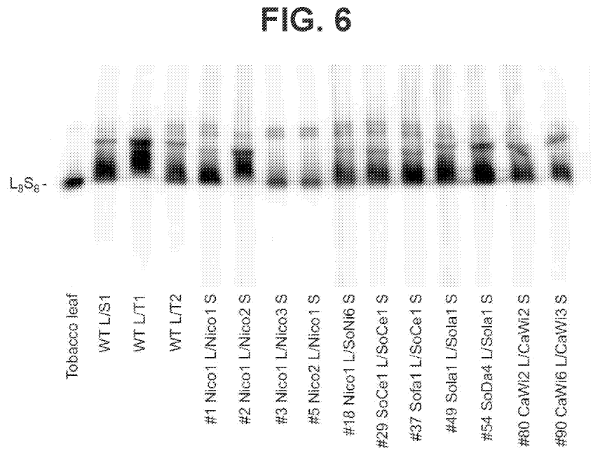

Just as in a previous study (Lin et al., Nat. Plants, 6: 1289-1299, 2020), the tobacco L+S-T1 Rubisco produced from E. coli displayed a markedly lower k_{cat}, likely due to the non-optimal E. coli environment for its assembly (Table 5). Native polyacrylamide gel electrophoresis (PAGE) analysis of 11 predicted ancestors with both high and low catalytic rates from each of the four ancestral nodes shows that most had similar migration as the tobacco leaf control and L+S-S1 or L+S-T2 enzyme produced in E. coli (FIG. 6). Only ancestor #2 with Nico1 L and Nico2 S subunits and the tobacco L+S-T1 Rubisco migrated at a slightly slower rate. Both the Nico2 S subunit and the tobacco S-T1 subunit share the I7Y mutation, which could explain the reduced mobility of the ancestor #2. This did not lead to poor carboxylation catalysis for the ancestor #2 as in the tobacco L+S-T1 Rubisco (Table 5). A recent study on Arabidopsis Rubisco expressed in E. coli found that incomplete N-terminal processing of its L subunit led to about 20% lower k_{cat}(Ng et al., J. Biol. Chem., 295: 16427-16435, 2020). The status of the N-terminal processing of the L subunits in the enzymes expressed from E. coli in our study is not known, but no negative impact on the k_{cat} for the Rubiscos expressed from E. coli was observed except for the enzyme with the tobacco S-T1 subunit.

Next, the RuBP carboxylation rates were measured at 30° C. for six representative ancestors and the same control samples. Both k_{cat} and K_{M,air} values of all samples were higher at 30° than at 25° C., as expected (Table 4). All six ancestors displayed similar or higher activation energies (ΔHₐ) for k_{cat}/K_{M,air} than the reference WT L+S-S1 control, indicating that their catalysis potentially has a higher optimal temperature. This is not unexpected since these enzymes should be adapted to a hotter climate associated with elevated CO₂ more than 20 Ma.

TABLE 4
Summary of RuBP carboxylation kinetics at 25° C. and 30° C. for six representative ancestral
Rubiscos predicted for different Solanaceae nodes and wild-type tobacco enzymes with different S subunits.

| | k_{cat} (s⁻¹) | | | K_{M, air} (μM) | | | k_{cat}/K_{M, air} (μM⁻¹s⁻¹) | | |
|---|---|---|---|---|---|---|---|---|---|
| Rubisco sample | 25° C. | 30° C. | ΔHₐ | 25° C. | 30° C. | ΔHₐ | 25° C. | 30° C. | ΔHₐ |
| Native (Nicotiana | 3.4 ± 0.2 | 5.1 ± 0.2 | 60.0 | 18.8 ± 0.7 | 24.0 ± 1.0 | 36.6 | .183 ± .004 | .214 ± .017 | 23.8 |
| tabacum) | (.760) | (.214) | | (.150) | (.102) | | (.006) | (.785) | |
| Nt-L + Nt-S-S1 | 3.5 ± 0.3 | 4.9 ± 0.2 | 50.2 | 17.0 ± 1.4 | 22.6 ± 1.5 | 42.6 | .206 ± .006 | .219 ± .012 | 9.3 |
| (reference) | | | | | | | | | |
| Nt-L + Nt-S-T1 | 2.4 ± 0.2 | 3.8 ± 0.2 | 67.2 | 16.0 ± 1.6 | 22.8 ± 1.7 | 53.1 | .151 ± .012 | .167 ± .019 | 14.5 |
| | (.008) | (.001) | | (.440) | (.902) | | (.007) | (.035) | |
| Nt-L + Nt-S-T2 | 3.4 ± 0.2 | 4.9 ± 0.2 | 54.9 | 17.7 ± 2.1 | 22.5 ± 0.6 | 35.6 | .193 ± .020 | .217 ± .003 | 18.1 |
| | (.563) | (.889) | | (.761) | (.772) | | (.563) | (.984) | |
| #1 Nico1 L + Nico1 S | 4.1 ± 0.2 | 5.6 ± 0.6 | 47.2 | 18.3 ± 1.3 | 23.5 ± 3.2 | 37.6 | .225 ± .004 | .241 ± .021 | 10.3 |
| | (.052) | (.162) | | (.333) | (.688) | | (.013) | (.176) | |
| #5 Nico2 L + Nico1 S | 4.4 ± 0.1 | 5.9 ± 0.3 | 46.2 | 18.9 ± 0.5 | 22.9 ± 1.6 | 28.4 | .231 ± .002 | .261 ± .007 | 18.0 |
| | (.030) | (.013) | | (.141) | (.835) | | (.012) | (<.001) | |
| #18 Nico1 L+ SoNi6 S | 4.2 ± 0.0 | 6.0 ± 0.2 | 53.5 | 18.4 ± 0.6 | 24.3 ± 1.6 | 42.2 | .230 ± .005 | .248 ± .013 | 11.5 |
| | (.049) | (.005) | | (.243) | (.198) | | (.007) | (.035) | |
| #37 Sofa1 L + SoCe1 S | 3.7 ± 0.1 | 5.6 ± 0.3 | 61.4 | 17.7 ± 2.5 | 24.2 ± 1.0 | 46.5 | .214 ± .022 | .233 ± .004 | 13.3 |
| | (.299) | (.034) | | (.711) | (.101) | | (.632) | (.002) | |
| #49 Sola1 L + Sola1 S | 4.1 ± 0.1 | 5.8 ± 0.5 | 50.3 | 19.2 ± 1.4 | 22.4 ± 2.7 | 23.3 | .217 ± .011 | .260 ± .009 | 27.1 |
| | (.040) | (.082) | | (.141) | (.885) | | (.218) | (.003) | |
| #80 CaWi2 L + CaWi2 S | 3.7 ± 0.2 | 5.6 ± 0.3 | 61.0 | 17.2 ± 0.6 | 22.6 ± 0.5 | 41.3 | .218 ± .008 | .248 ± .006 | 19.7 |
| | (.591) | (.025) | | (.878) | (.997) | | (.120) | (.001) | |

Means ± SD (P-values) of k_{cat}, K_{M, air} and k_{cat}/K_{M, air} obtained from three E. coli or leaf soluble extracts (n = 3) for each sample are shown. The P-values compared to the measurements from the tobacco enzyme with L and S-S1 subunits were determined with two-tailed heteroscedastic t-tests. ΔHₐ values are in kJ⁻¹ mol⁻¹.

TABLE 5
Summary of RuBP carboxylation kinetics at 25° C. for 38 ancestral Rubiscos predicted
for different Solanaceae nodes and wild-type tobacco enzymes with different S subunits.

| | k_{cat} (s⁻¹) | | K_{M, air} (μM) | | k_{cat}/K_{M, air} (μM⁻¹s⁻¹) | |
|---|---|---|---|---|---|---|
| Rubisco sample | Mean ± SD | P-value | Mean ± SD | P-value | Mean ± SD | P-value |
| Native tobacco enzyme | 3.4 ± 0.2 | 0.760 | 18.8 ± 0.7 | 0.150 | 0.183 ± 0.004 | 0.006 |
| Nt-L + Nt-S-S1 | 3.5 ± 0.3 | Reference | 17.0 ± 1.4 | Reference | 0.206 ± 0.006 | Reference |
| Nt-L + Nt-S-T1 | 2.4 ± 0.2 | 0.008 | 16.0 ± 1.6 | 0.440 | 0.151 ± 0.012 | 0.007 |
| Nt-L + Nt-S-T2 | 3.4 ± 0.2 | 0.563 | 17.7 ± 2.1 | 0.761 | 0.193 ± 0.020 | 0.563 |
| #1 Nico1 L + Nico1 S | 4.1 ± 0.2 | 0.052 | 18.3 ± 1.3 | 0.333 | 0.225 ± 0.004 | 0.013 |
| #2 Nico1 L + Nico2 S | 3.5 ± 0.1 | 0.883 | 16.8 ± 1.2 | 0.841 | 0.208 ± 0.001 | 0.815 |
| #5 Nico2 L + Nico1 S | 4.4 ± 0.1 | 0.030 | 18.9 ± 0.5 | 0.141 | 0.231 ± 0.002 | 0.012 |
| #9 Nico3 L + Nico1 S | 4.1 ± 0.1 | 0.065 | 20.1 ± 1.8 | 0.083 | 0.203 ± 0.019 | 0.797 |
| #13 Nico1 L + SoNi1 S | 4.1 ± 0.2 | 0.043 | 21.2 ± 2.5 | 0.080 | 0.197 ± 0.018 | 0.471 |
| #17 Nico1 L + SoNi5 S | 4.4 ± 0.2 | 0.018 | 20.8 ± 1.5 | 0.036 | 0.212 ± 0.014 | 0.574 |
| #18 Nico1 L + SoNi6 S | 4.2 ± 0.0 | 0.049 | 18.4 ± 0.6 | 0.243 | 0.230 ± 0.005 | 0.007 |
| #19 Nico1 L + SoNi7 S | 4.1 ± 0.1 | 0.058 | 18.3 ± 0.8 | 0.278 | 0.227 ± 0.009 | 0.036 |
| #20 Nico1 L + SoNi8 S | 4.2 ± 0.2 | 0.030 | 20.9 ± 0.3 | 0.039 | 0.203 ± 0.010 | 0.642 |
| #23 Nico2 L + SoNi3 S | 4.0 ± 0.0 | 0.093 | 17.4 ± 1.2 | 0.751 | 0.231 ± 0.014 | 0.075 |
| #27 Nico2 L + SoNi7 S | 4.0 ± 0.1 | 0.089 | 18.3 ± 1.2 | 0.320 | 0.220 ± 0.010 | 0.124 |
| #28 Nico2 L + SoNi8 S | 3.9 ± 0.2 | 0.109 | 16.8 ± 1.6 | 0.850 | 0.236 ± 0.014 | 0.054 |
| #91 Nico3 L + SoNi1 S | 3.5 ± 0.3 | 0.991 | 18.1 ± 3.4 | 0.648 | 0.197 ± 0.022 | 0.538 |
| #93 Nico3 L + SoNi3 S | 3.6 ± 0.2 | 0.745 | 18.2 ± 1.1 | 0.347 | 0.198 ± 0.017 | 0.500 |
| #94 Nico3 L + SoNi4 S | 3.5 ± 0.2 | 0.889 | 18.9 ± 1.0 | 0.143 | 0.187 ± 0.010 | 0.057 |
| #97 Nico3 L + SoNi7 S | 3.8 ± 0.2 | 0.226 | 18.0 ± 1.5 | 0.473 | 0.212 ± 0.010 | 0.440 |
| #98 Nico3 L + SoNi8 S | 3.9 ± 0.2 | 0.123 | 18.4 ± 1.1 | 0.272 | 0.214 ± 0.009 | 0.261 |
| #37 Sofa1 L + SoCe1 S | 3.7 ± 0.1 | 0.299 | 17.7 ± 2.5 | 0.711 | 0.214 ± 0.022 | 0.632 |
| #38 Sofa1 L + SoCe2 S | 3.9 ± 0.2 | 0.126 | 18.3 ± 2.7 | 0.512 | 0.216 ± 0.023 | 0.557 |
| #39 Sofa1 L + SoCe3 S | 3.9 ± 0.2 | 0.134 | 18.6 ± 2.3 | 0.385 | 0.211 ± 0.019 | 0.684 |
| #40 Sofa1 L + SoCe4 S | 3.8 ± 0.3 | 0.313 | 17.5 ± 2.8 | 0.803 | 0.218 ± 0.018 | 0.399 |
| #49 Sola1 L + Sola11 S | 4.1 ± 0.1 | 0.040 | 19.2 ± 1.4 | 0.141 | 0.217 ± 0.011 | 0.218 |
| #50 Sola2 L + Sola11 S | 4.2 ± 0.3 | 0.040 | 19.7 ± 0.9 | 0.065 | 0.212 ± 0.004 | 0.216 |
| #54 SoDa4 L + Sola11 S | 3.2 ± 0.2 | 0.211 | 17.1 ± 3.0 | 0.990 | 0.191 ± 0.024 | 0.396 |
| #55 Sola1 L + Sola2 S | 4.1 ± 0.2 | 0.058 | 18.7 ± 1.7 | 0.281 | 0.219 ± 0.011 | 0.171 |
| #58 SoDa2 L + Sola2 S | 3.3 ± 0.2 | 0.349 | 17.2 ± 1.0 | 0.912 | 0.192 ± 0.006 | 0.046 |
| #60 SoDa4 L + Sola2 S | 3.3 ± 0.2 | 0.342 | 18.0 ± 0.9 | 0.375 | 0.183 ± 0.004 | 0.006 |
| #61 Sola1 L + Sola3 S | 4.0 ± 0.2 | 0.071 | 17.6 ± 1.2 | 0.652 | 0.230 ± 0.008 | 0.016 |
| #62 Sola2 L + Sola3 S | 4.2 ± 0.1 | 0.034 | 18.5 ± 0.7 | 0.211 | 0.228 ± 0.010 | 0.038 |
| #63 SoDa1 L + Sola3 S | 3.3 ± 0.1 | 0.309 | 16.7 ± 1.8 | 0.820 | 0.198 ± 0.022 | 0.581 |
| #65 SoDa3 L + Sola3 S | 3.6 ± 0.2 | 0.686 | 17.7 ± 0.9 | 0.535 | 0.203 ± 0.002 | 0.487 |
| #67 Sola1 L + SoJa1 S | 4.1 ± 0.1 | 0.059 | 18.5 ± 0.6 | 0.214 | 0.222 ± 0.006 | 0.030 |
| #68 Sola2 L + SoJa1 S | 4.0 ± 0.1 | 0.074 | 18.1 ± 0.4 | 0.329 | 0.223 ± 0.012 | 0.124 |
| #79 CaWi1 L + CaWi2 S | 3.6 ± 0.2 | 0.591 | 17.4 ± 2.7 | 0.836 | 0.211 ± 0.026 | 0.774 |
| #80 CaWi2 L + CaWi2 S | 3.7 ± 0.2 | 0.328 | 17.2 ± 0.6 | 0.878 | 0.218 ± 0.008 | 0.120 |
| #83 CaWi5 L + CaWi2 S | 3.7 ± 0.0 | 0.437 | 16.8 ± 0.6 | 0.830 | 0.219 ± 0.010 | 0.164 |
| #86 CaWi2 L + CaWi3 S | 3.7 ± 0.3 | 0.405 | 18.7 ± 3.1 | 0.460 | 0.201 ± 0.017 | 0.680 |
| #88 CaWi4 L + CaWi3 S | 3.3 ± 0.2 | 0.278 | 15.5 ± 0.6 | 0.205 | 0.210 ± 0.015 | 0.702 |

Means ± SD of k_{cat}, K_{M, air} and k_{cat}/K_{M, air} obtained from three E. coli or leaf soluble extracts (n = 3) for each sample are shown. The P-values compared to the measurements from the tobacco enzyme with L and S-S1 subunits were determined with two-tailed heteroscedastic t-tests.

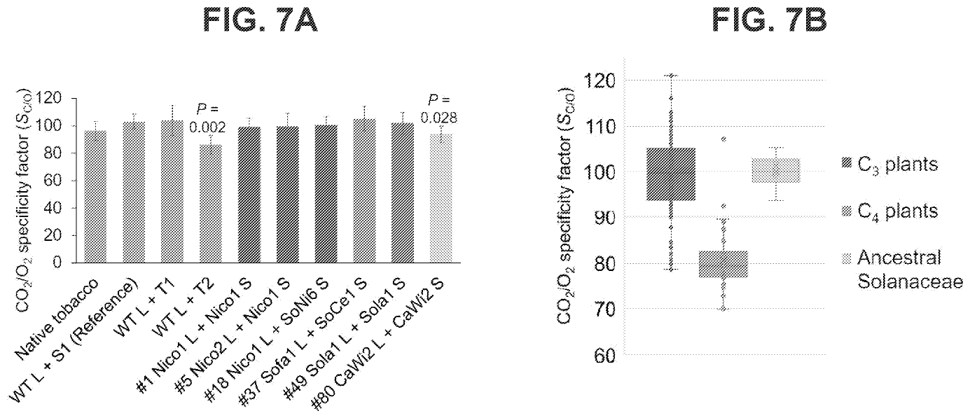

C₄ Rubiscos typically have lower CO₂/O₂ specificity factors (S_{C/O}) compared to C₃ versions (Sharwood et al., Nat. Plants, 2: 16186, 2016; Flamholz et al., Biochemistry, 58: 3365-3376, 2019; Cummins et al., Front. Plant Sci., 12: 662425, 2021). Since many ancestors predicted here have similar k_{cat} as C₄ Rubiscos, it was tested whether they are also associated with similar S_{C/O} as C₄ enzymes. Six representative ancestral enzymes were partially purified and their Scio was measured at 25° C. Surprisingly, the S_{C/O} values of five ancestors are statistically similar to that of the tobacco WT L+S-S1 control. Only one predicted ancestor (#80 CaWi2 L+CaWi2 S) and the tobacco WT L+S-T2 sample had somewhat lower S_{C/O} (FIG. 7A). Comparison to the previously reported S_{C/O} values of C₃ and C₄ enzymes also indicates that these six ancestors were able to distinguish CO₂ from O₂ as efficiently as the C₃ enzymes (FIG. 7B).

### (d) Discussion

The present study overcomes the lack of available Rubisco sequences, especially for the S subunits, with de novo assembly from transcriptomics data. The workflow presented herein is computationally efficient and capable of removing most, if not all, chimeric assemblies and can generally be applied to any gene of interest. In fact, errors in several NCBI records were identified, mostly generated from early periods when DNA sequencing was tedious and had low accuracy.

The ancestral Rubiscos of Solanaceae predicted in this study appear to be robust, thermally stable, and represent great candidates for evolutionary studies. Several enzymes with higher k_{cat} and efficiency in each of the four ancestral groups were identified, indicating that all of these enzymes probably evolved at higher CO₂ levels. The best enzymes were identified among Nico and Sola ancestral groups, potentially due to higher accuracy in their predicted sequences enabled by the overrepresentation of extant Solanum and Nicotiana sequences used in the present phylogenetic analyses. Despite the relatively small numbers of residue substitutions with no apparent alteration in their overall polarity or electrostatic properties, the subtle mutations in many of these predicted ancestors were able to capture important kinetic traits likely possessed by the actual ancestors. Notably, the majority of the predicted ancestors have more mutations in the S subunits than in the L subunits although the S subunits are only one-fourth the size of the L subunits and are not directly involved in catalysis. A recent study found that the kinetics of potato Rubisco expressed in tobacco were significantly affected by the identity of the S subunit (Martin-Avila et al., Plant Cell, 32: 2898-2916, 2020). This is consistent with the present findings that show that many of the predicted ancestors have extant L subunits and yet are able to perform the catalysis more efficiently than the extant enzymes, indicating that the ancestral S subunits in them likely influence the kinetics positively. However, none of the predicted ancestors with enhanced carboxylation abilities contains either of the two unique amino acid residues identified in the S subunit of the potato Rubisco with higher k_{cat} and efficiency (Martin-Avila et al., Plant Cell, 32: 2898-2916, 2020). This highlights the difficulty of predicting the key residues that might control the kinetic properties and the importance of considering both subunits simultaneously to optimize the assembly and overall rigor of the enzyme.

Residue substitutions at 145, 219, 225, 279, 439, and 449 in the L subunits of the predicted ancestors were previously identified to be positively selected during the evolution of Rubiscos in plants (Kapralov and Filatov, BMC Evol. Biol., 7: 73, 2007), and the L225I substitution in most of the predicted ancestral L subunits of Solanaceae is consistent with the 1225L substitution previously found to be associated with the evolution of C₃ Rubiscos (Studer et al., Proc. Natl. Acad. Sci. U.S.A., 111: 2223-2228, 2014). It is not unexpected that none of the substitutions in the predicted ancestors was found to be involved in the transition from C₃ to C₄ photosynthesis (10) since C₄ photosynthesis is not present in Solanaceae. Because the residues altered in both subunits of the ancestors are not directly associated with those at the active site, it is challenging to decipher how the residue substitutions in the predicted ancestral Rubiscos were able to influence the kinetic properties without further structural studies.

In some families with both C₃ and C₄ photosynthesis, the C₃ Rubiscos have lower Scio than the average Scio of typical C₃ Rubiscos, which likely facilitated the evolution of C₄ photosynthesis in those families (Cummins et al., Front. Plant Sci., 12: 662425, 2021). In contrast, the ancestral C₃ Rubiscos of Solanaceae predicted here have similar Scio as typical C₃ Rubiscos. Interestingly, recent structural analyses indicated a correlation between Scio and positively charged cavities close to the active site (Poudel et al., Proc. Natl. Acad. Sci. U.S.A., 117: 30541-30547, 2020). Based on the residue substitutions, most of the predicted Solanaceae ancestors are expected to have similar electrostatic profiles as typical C₃ Rubiscos. Nevertheless, the present findings support the hypothesis that the catalytic behavior of C₃ Rubiscos in ancient plants prior to the emergence of C₄ photosynthesis may be more similar to the present day C₄ Rubiscos in having higher k_{cat}. The evolution of C₄ photosynthesis likely shifted their Rubiscos' S_{C/O} and affinity for CO₂ lower, while the enzymes remaining in C₃ plants shifted their k_{cat} lower during their adaptation to decreasing CO₂ levels. A previous study on the C₃ and C₄ L subunits in Flaveria species identified residue 309 as the catalytic switch, which is specific to the Flaveria species and incompatible with the tobacco L subunit background (Whitney et al., Proc. Natl. Acad. Sci. U.S.A., 108: 14688-14693, 2011). Multiple ancestral L and S subunits of Solanaceae characterized in this study were able to achieve the high catalytic rates of C₄ enzymes without sacrificing affinity for CO₂. It is also noteworthy that these ancestral subunits are highly similar to the tobacco sequences and are expected to be compatible with the Rubisco assembly system of tobacco chloroplasts. The present approach can be applied to study Rubiscos in other families of higher plants, especially the ones that include C₄ members, to investigate whether their ancestral Rubiscos display comparable features.

Higher catalytic efficiency of Rubisco is beneficial not only for growth, but also for water and nitrogen use efficiency in plants. The ancestral Rubiscos predicted in this study also appear adapted to hotter and drier environments based on their catalysis at a higher temperature and Scio values that are similar to the current C₃ Rubiscos. The next step will be to introduce these ancestral Rubiscos into plants and assess their performance. Although the technology to replace both Rubisco subunits was recently reported for tobacco (Martin-Avila et al., Plant Cell, 32: 2898-2916, 2020), transformed plants must be able to produce sufficient amount of Rubisco in order to take advantage of improved kinetics. Emerging technologies such as targeted base editing of chloroplast genes (Nakazato et al., Nat. Plants, 7: 539, 2011) should expand the engineering of Rubisco to other plants where generation of stable chloroplast transformation is not available. The procedure in this study can be a blueprint to identify superior Rubiscos in other families to eventually enhance carbon fixation in agricultural crops such as rice and wheat.

### Materials and Methods:

### De Novo Assembly of Sequences Encoding Rubisco Subunits

Each SRA file was downloaded with fastq-dump 2.8.0 program available from SRA Toolkit. The SRA file's reads aligned to sequences encoding Rubisco L or S subunits were selected with BBMap 38.22-1 program (by Bushnell B) using the DNA sequences encoding tobacco L subunit or the mature S subunit S1 as references in “vslow” and “local” modes and “maxindel” set to 100. Next, the paired reads in the fastq file exported by BBMap were separated into two fastq files with BBMap's bbsplitpairs scripts. Reads in the two fastq files were then assembled de novo by Trinity 2.8.5 three separate times as follows: (i) -KMER_SIZE 32; (ii) stringent setting, which includes “-min_kmer_cov 4-min_glue 4 -min_iso_ratio 0.2 -glue_factor 0.2 -jaccard_clip”; and (iii) both -KMER_SIZE 32 and stringent setting. If there were more than 10,000 reads in each fastq file, the first 5000 reads extracted by seqtk 1.3-r106 program were assembled in two more Trinity runs with -KMER_SIZE 32 with or without the stringent setting. The read coverages of starting bases for coding sequences were then obtained for assemblies that covered at least 90% of the reference sequences with alignment scores greater than 350 using BBMap scripts with “perfectmode” and “startcov=t” settings. The above process was automated with Python scripts (FIG. 1A), which were executed in Windows Subsystem for Linux from a shell script file, which can be supplied with multiple SRA IDs for high-throughput assembly. The scripts were written for the paired-end format of SRA files, although they can be adapted for single-end format with slight modifications. The automated process wrote SRA IDs, reference files used in BBMap, assembled sequences, sequences encoding the L and S subunits of Rubisco, and locations for the read coverage files of all assemblies to a csv file. In addition, it also saved read coverage files and PNG format images of read coverage profiles for the assemblies. In the first clean-up step, the read coverage images were visually inspected for gaps to remove chimeric assemblies. In the second clean-up step, assemblies generated for each species were compared against one another for the presence of long overlaps, and those that have long overlaps and were assembled at lower frequencies were removed.

RNA-Seq of Partial rbcS Transcripts

The seeds for Browallia viscosa (Bv), Nicandra physalodes (Np), Schizanthus coccineus (Sc), Schizanthus grahamii (Sg), and Vestia lyciodes (VI) were obtained from Plant World Seeds, and Anthocercis littorea (Al), Fabiana imbricata (Fi), and Jaborosa sativa (Js) were obtained from B & T World Seeds. DNA oligonucleotides were synthesized by Integrated DNA Technologies Inc. (Coralville, IA, USA). An Invitrogen PureLink RNA mini kit (Thermo Fisher Scientific Inc.) was used to prepare RNA samples from leaf tissues of plants grown under 100 photosynthetically active radiation (μmol/m2 per second) with a 16-hour photoperiod in Lambert LM-111 all-purpose mix. Invitrogen SuperScript III First-Strand Synthesis Supermix (Thermo Fisher Scientific Inc.) was used to synthesize cDNA with the Not I-dT-R oligonucleotide according to the manufacturer's instructions. Partial rbcS transcripts were amplified from each cDNA sample by Phusion high-fidelity DNA polymerase with Not I-Adpr-R and Mau BI-SSU-D-F oligonucleotides, and ˜650-base pair (bp) amplicons were extracted from agarose gels with an EZ-10 spin-column polymerase chain reaction (PCR) product purification kit (Thermo Fisher Scientific Inc.). Bv, Np, Sc, Sg, and VI samples were fragmented with Covaris E220 followed by reparation and adenylation of ends and adapter ligation with a TruSeq DNA PCR-Free kit (Illumina Inc.) before they were pooled and sequenced with NextSeq 550 (Illumina Inc.) in 2×150-bp runs. Np, Al, Fi, and Js samples were fragmented and indexed with a Nextera DNA library prep kit (Illumina Inc.) and sequenced with MiSeq nano (Illumina Inc.) in 2×250-bp runs.

### Predicting Ancestral Rubisco Sequences

Multiple sequence alignments of the Rubisco L and S subunits were performed with Clustal Omega 1.2.4 (Sievers et al., Mol. Syst. Biol., 7: 539, 2011). Bayesian inference was performed separately with MrBayes 3.2.7a (Ronquist et al, Syst. Biol., 61: 539-542, 2012) using the amino acid sequences of the L and S subunits with the following parameters: Iset nst=mixed rates=invgamma, prset aamodelpr=mixed, mcmc ngen=600,000 for L subunits or 800,000 for S subunits, temp=0.06 for L subunits or 0.04 for S subunits, startparams=reset, and starttree=random. The topology was fixed at multiple nodes based on the reported consensus tree (Sarkinen et al., BMC Evol. Biol., 13: 214, 2013), and the probabilities of the ancestral states at those nodes were generated with the setting “report applyto=(1) ancstates=yes.” The average SDs of split frequencies from Metropolis-coupled Markov chain Monte Carlo sampling bottomed at about 0.02. The ancestral states were also estimated with RAxML 8.2 (Stamatakis et al., Bioinformatics, 30:1312-1313, 2014) with PROTGAMMAAUTO for model configuration, autoMRE for rapid bootstrapping with automatic criteria, “-g” option with a constraint tree file to ensure the topology remained consistent with the established tree (Sarkinen et al., BMC Evol. Biol., 13: 214, 2013), and “-f A” setting with the resulting best tree rooted with FigTree program v1.4.3. The phylogenies of L and S subunits reached convergence after 650 and 750 bootstrap replicates, respectively. From the predicted probabilities at each residue position of eight selected nodes (Table 2), 98 combinations of ancestral L and S subunits (Table 3) were selected.

Expressing the Predicted Ancestral Rubiscos in E. coli

DNA oligonucleotides were purchased from Integrated DNA Technologies Inc. (Coralville, IA, USA). Phusion high-fidelity DNA polymerase, FastDigest restriction enzymes, and T4 DNA ligase were purchased from Thermo Fisher Scientific Inc. and used to amplify, digest, and ligate DNA fragments. Mau BI site was inserted before T7P-lacO- RBS-Nt-rbcL operon by amplifying the operon with Mlu I-Age I-Mau BI-for and BJFEseqR oligonucleotides from BJFE-T7P-lacO- RBC-Nt-rbcL plasmid (Lin et al., Nat. Plants, 6: 1289-1299, 2020), which was then digested with Mlu I and Not I and ligated into the Mlu I and Not I sites of a holding vector to obtain pHD-T7P-NtL vector. Next, T7P-lacO-RBC-NtrbcL operon digested from pHD-T7P-NtL with Age I was ligated into the Age I site of pAtC60αβ/C20 (Aigner et al., Science, 358: 1272-1278, 2017) vector to obtain pET-AtC60AB20-T7P-NtL-v2 vector. The L subunit gene was separated into three fragments based on the two internal restriction sites: Bam HI at residue 155 and Nde I at residue 387. The mutations in the predicted ancestral L subunits (Table 3) were introduced with overlapping PCRs by corresponding oligonucleotides and accumulated in each of the three fragments, which were then simultaneously ligated into Mau BI and Not I sites of pET-AtC60AB20-T7P-NtL-v2 vector to generate the final expression vectors. The tobacco S subunit T2 gene was separated into two fragments at Eco RI restriction site located at residues 43 to 44 and used as the template to generate the predicted ancestral S subunits (Table 3). Substitutions at residues 23, 28, 30, 85, 88, and 96 were achieved by overlapping PCRs, while the remaining substitutions were generated with a Q5 site-directed mutagenesis kit (New England Biolabs) with the corresponding oligonucleotides. The mutations accumulated in each of the two fragments were combined by ligation into Nco I and Not I sites of pCDF-NtXT2R1AtR2NtB2 vector (Lin et al., Nat. Plants, 6: 1289-1299, 2020) to obtain the final expression vectors. The sequence of each ligated DNA in the expression vectors was confirmed by Sanger sequencing. The pET-AtC60AB20-T7P- NtL-v2 and pCDF-NtXT2R1AtR2NtB2 vectors were cotransformed into BL21*(DE3) E. coli, and each Rubisco sample was expressed from the E. coli culture grown in ZYP-5052 autoinduction medium as described previously (Lin et al., Nat. Plants, 6: 1289-1299, 2020).

### Enzyme Kinetics of the Predicted Ancestral Rubiscos

Soluble extracts from 6-ml E. coli cultures lysed in 400 μl of 50 mM tris-HCl (pH 8), 10 mM MgCl₂, 1 mM EDTA, 20 mM NaHCO₃, 2 mM dithiothreitol (DTT), and Pierce protease inhibitor minitablet (Thermo Fisher Scientific Inc.) were used to measure RuBP carboxylation activities of the Rubisco samples. For leaf extracts, about 5 cm² of leaf tissue each suspended in 500 μl of 100 mM Bicine-NaOH (pH 7.9), 5 mM MgCl2, 1 mM EDTA, 5 mM ε-aminocaproic acid, 2 mM benzamidine, 50 mM 2-mercaptoethanol, protease inhibitor cocktail, 1 mM phenylmethanesulfonyl fluoride, 5% (w/v) poly(ethylene glycol) 4000, 10 mM NaHCO3, and 10 mM DTT was crushed in a 2-ml Wheaton homogenizer for about 1 min on ice, and insoluble materials were removed by centrifugation at 16,000 rcf at 4° C. for 5 min. Each supernatant of leaf extracts was then applied to a 2-ml Zeba spin de-salting column with 40,000 molecular weight cutoff preequilibrated with 100 mM Bicine-NaOH (pH 8), 20 mM MgCl2, 1 mM EDTA, 1 mM benzamidine, 1 mM ε-aminocaproic acid, 1 mM KH₂PO₄, 2% (w/v) poly(ethylene glycol) 4000, 20 mM NaHCO3, 10 mM DTT, and each eluate following centrifugation at 1000 rcf at 4° C. for 2 min was incubated at 23° C. for 30 min for full activation of Rubisco active sites. RuBP carboxylation experiments were performed as described previously with NaH₁₄CO₃ solutions with different concentrations and specific activities, such that 14C activities of acid-stable compounds in the vials following the termination of the reactions gave a similar range of values (Lin et al., Nat. Plants, 6: 1289-1299, 2020). For initial screening of the 98 predicted ancestral enzymes, RuBP carboxylation activities were measured in vials equilibrated with N₂ gas at 25° C. and 108 μM [CO2], and ¹⁴C fixed to stable organic compounds was counted with Tri-Carb 2810TR Scintillation counter (PerkinElmer). The same Rubisco samples were used for quantification of Rubisco active sites on the same day with 14C-carboxyarabinitol bisphosphate (CABP) bound to each sample as described previously (Lin et al., Nat. Plants, 6: 1289-1299, 2020). The specific activity of ¹⁴C CABP was precalibrated with a soluble extract from spinach leaf tissue, where the Rubisco concentration was determined from an immunoblot along with a commercial spinach RbcL standard (Agrisera, part no. AS01 017S) using a polyclonal antibody against wheat Rubisco (Lin et al., Nat. Plants, 6: 1289-1299, 2020). To measure kcat and KM,ₐᵢᵣ, the RuBP carboxylation activities of E. coli soluble extracts with 38 predicted ancestral Rubiscos and three tobacco Rubiscos and soluble extracts from tobacco leaf tissue were measured at six different [CO2] concentrations ranging from 5.5 to 90 μM at pH 8 in vials equilibrated with CO₂-free air at 25° C., and the Rubisco active sites were subsequently quantified with ¹⁴C CABP. kcat and KM,ₐᵢᵣ were obtained from nonlinear least square fitting to the classical Michaelis-Menton equation as described previously (Lin et al., Nat. Plants, 6: 1289-1299, 2020). Three biological replicates were performed for each sample from three separate E. coli cultures or leaf extracts. The same measurements were repeated at 30° C. for six predicted ancestral Rubisco samples and the same control samples of tobacco Rubiscos.

### Specificity Factors of the Predicted Ancestral Rubiscos

CO₂/O₂ specificity factors (S_{C/O}) of six predicted ancestral Rubiscos and tobacco Rubiscos were measured with partially purified Rubisco samples. First, E. coli pellets from 1.5- to 2-liter cultures were each resuspended in ˜20 ml of extraction buffer [25 mM triethanolamine (pH 8), 5 mM MgCl₂, 0.5 mM EDTA, 1 mM KH₂PO₄, 1 mM benzamidine, 5 mM ε-aminocaproic acid, 10 mM 2-mercaptoethanol, 5 mM NaHCO₃, 2 mM DTT, and 1 mM phenylmethylsulfonyl fluoride] and sonicated with eight 10-s pulses over 5 min at 4° C. Insoluble materials were separated with centrifugation at 35,000 g at 4° C. for 30 min. The supernatant was applied to a 5-ml HiTrap Q HP anion exchange column (GE Healthcare) connected to the ÄKTA P-900 Fast Protein Liquid Chromatography System equipped with an Inv-907 valve and a Frac-950 fraction collector and equilibrated with Q buffer [25 mM triethanolamine (pH 8), 5 mM MgCl2, 0.5 mM EDTA, 1 mM benzamidine, 1 mM ε-aminocaproic acid, 5 mM NaHCO₃, 2 mM DTT, and 12.5% (v/v) glycerol]. NaCl in the buffer applied to the column was then increased from 0 to 0.5 M over 75 ml of volume at 2 ml min⁻¹, and the eluents were collected in 2-ml fractions. The Rubisco-containing fractions were identified by bound ¹⁴C CABP, concentrated to ˜500 to 700 μl with Amicon Ultra-15 centrifugal filter units, and stored at −80° C. before use. Rubisco was also purified with the 5-ml HiTrap Q HP column from ˜500 cm² of tobacco leaf tissue broken in ˜200 ml of extraction buffer in a blender, precipitated with PEG at a final concentration of ˜20% (w/v), and resuspended in ˜10 ml of Q buffer. Total protein concentration in the samples was estimated with Bradford assays. The Rubisco purified from tobacco leaf tissue represented about 90% of the total soluble protein, while the Rubisco samples from E. coli represented about 25 to 30% of the total soluble protein. The Scio values were calculated with the formula (RuBP carboxylated/RuBP oxygenated)/([CO₂]/[O₂]) after measuring RuBP carboxylated at three different ratios of [CO₂]/[O₂] (Parry et al., J. Exp. Bot., 40: 317-320, 1989). The amount of RuBP oxygenated was derived from the total RuBP consumed in each experiment. After ˜25 nmol of RuBP was entirely catalyzed by ˜140 pmol of Rubisco active sites at three [CO₂] concentrations in each reaction vial equilibrated with CO₂-free air at 25° C., the ¹⁴C fixed to stable organic compounds was counted. Each reaction was also repeated in a second vial with 2 min of additional incubation period to ensure that all RuBP was consumed in both measurements. In addition, each reaction was repeated in a vial equilibrated with N₂ gas, from which the total amount to RuBP consumed in each vial was obtained, since all RuBP was carboxylated in these vials.

### Native PAGE and Immunoblot

Soluble extracts were prepared from either E. coli cultures or tobacco leaf tissue in the same procedure as in the determination of Rubisco kinetics as described above. The total soluble protein concentrations were determined with Bradford assays, and 4 μg of total soluble proteins from each E. coli extract or 0.1 μg from tobacco leaf extract was mixed with the loading buffer made up of 50 mM bis-tris (pH 7.2), 50 mM NaCl, 0.001% Ponceau S, and 10% glycerol. The electrophoresis was carried out in an Invitrogen 3 to 15% bis-tris protein gel from Thermo Fisher Scientific with 50 mM bis-tris and 50 mM tricine (pH 6.8) anode buffer and 0.002% Coomassie Brilliant Blue G250, 50 mM bis-tris, and 50 mM tricine (pH 6.8) cathode buffer at 150 V and 4° C. for 30 min followed by 250 V for 60 min. The samples were then transferred to a polyvinylidene difluoride membrane with 0.45-μm pore size in 25 mM tris, 192 mM glycine, and 20% methanol at 100 V and 4° C. for 1 hour. The membrane was blocked with 5% milk in TBST (tris-buffered saline with Tween 20) buffer [20 mM tris (pH 7.5), 150 mM NaCl, and 0.1% Tween 20] at 23° C. for 1 hour, incubated with an antibody against Rubisco (from P. J. Andralojc from Rothamsted Research, raised in a rabbit) in 5% milk in TBST buffer at 4° C. overnight, and detected with horseradish peroxidase-conjugated secondary antibody in 2.5% milk in TBST buffer at 23° C. for 1 hour. The chemiluminescent signals from enhanced chemiluminesence substrate were captured with a ChemiDoc MP imaging system from Bio-Rad.

### OTHER EMBODIMENTS

Some embodiments of the technology described herein can be defined according to any of the following numbered embodiments:

A1. A Rubisco enzyme complex comprising:
a recombinant amino acid sequence comprising an amino acid sequence having at least 80%, 85%, 90%, 95%, 98% sequence identity to SEQ ID NO: 1-19.

A2. A Rubisco enzyme complex comprising:
a recombinant amino acid sequence comprising an amino acid sequence having at least 80%, 85%, 90%, 95%, 98% sequence identity to SEQ ID NO: 20-42.

A3. A Rubisco enzyme complex comprising:
a recombinant first amino acid sequence comprising an amino acid sequence having at least 80%, 85%, 90%, 95%, 98% sequence identity to SEQ ID NO: 1-19, and
a recombinant second amino acid sequence comprising an amino acid sequence having at least 80%, 85%, 90%, 95%, 98% sequence identity to SEQ ID NO: 20-42.

A4. A Rubisco enzyme complex comprising:
a recombinant amino acid sequence comprising one or more point mutations as indicted in SEQ NO: 1-42.

B1. A recombinant Rubisco system comprising:
a nucleic acid sequence encoding an amino acid sequence having at least 80%, 85%, 90%, 95%, 98% sequence identity to SEQ ID NO: 1-19.

B2. A recombinant Rubisco system comprising:
a nucleic acid sequence encoding an amino acid sequence having at least 80%, 85%, 90%, 95%, 98% sequence identity to SEQ ID NO: 20-42.

B3. A recombinant Rubisco system comprising:
a nucleic acid sequence encoding an amino acid sequence having at least 80%, 85%, 90%, 95%, 98% sequence identity to SEQ ID NO: 1-19; and
a nucleic acid sequence encoding an amino acid sequence having at least 80%, 85%, 90%, 95%, 98% sequence identity to SEQ ID NO: 20-42.

B4. A Rubisco enzyme complex comprising:
a recombinant nucleic sequence encoding for one or more point mutations as indicted in SEQ NO: 1-42.

C₁. A method of identifying and engineering a Rubisco complex comprising one or more steps indicated in the Example.

D1. A genetically engineered plant comprising one or more of the amino acid sequences of claims A1-A4.

E1. A genetically engineered plant comprising one or more of the nucleic acid sequences of claims B1-B4.

1. A genetically engineered plant comprising:
(a) a Rubisco large subunit (LSU) comprising L225I and K429Q amino acid substitution mutations, wherein the amino acid substitution mutations are numbered relative to the LSU of Nicotiana tabacum (SEQ ID NO: 43); and
(b) a Rubisco small subunit (SSU) comprising N8G, V30I, and E88Q amino acid substitution mutations, wherein the amino acid substitution mutations are numbered relative to the S-T2 subunit of Nicotiana tabacum (SEQ ID NO: 44).

2. The genetically engineered plant of claim 1, wherein the Rubisco LSU comprises an amino acid sequence having at least 80%, 85%, 90%, 95%, 98%, or 99% sequence identity to SEQ ID NO: 1.

3. The genetically engineered plant of claim 2, wherein the Rubisco LSU comprises the amino acid sequence of SEQ ID NO: 1.

4. The genetically engineered plant of claim 1, wherein the Rubisco SSU comprises an amino acid sequence having at least 80%, 85%, 90%, 95%, 98%, or 99% sequence identity to SEQ ID NO: 20.

5. The genetically engineered plant of claim 4, wherein the Rubisco SSU comprises the amino acid sequence of SEQ ID NO: 20.

6. A genetically engineered plant comprising:
(a) a Rubisco LSU comprising V145I, L225I, and K429Q amino acid substitution mutations, wherein the amino acid substitution mutations are numbered relative to the LSU of Nicotiana tabacum (SEQ ID NO: 43); and
(b) a Rubisco SSU comprising N8G, V30I, and E88Q amino acid substitution mutations, wherein the amino acid substitution mutations are numbered relative to the S-T2 subunit of Nicotiana tabacum (SEQ ID NO: 44).

7. The genetically engineered plant of claim 6, wherein the Rubisco LSU comprises an amino acid sequence having at least 80%, 85%, 90%, 95%, 98%, or 99% sequence identity to SEQ ID NO: 2.

8. The genetically engineered plant of claim 7, wherein the Rubisco LSU comprises the amino acid sequence of SEQ ID NO: 1.

9. The genetically engineered plant of claim 6, wherein the Rubisco SSU comprises an amino acid sequence having at least 80%, 85%, 90%, 95%, 98%, or 99% sequence identity to SEQ ID NO: 20.

10. The genetically engineered plant of claim 9, wherein the Rubisco SSU comprises the amino acid sequence of SEQ ID NO: 20.

11. A genetically engineered plant comprising:
(a) a Rubisco LSU comprising L225I and K429Q amino acid substitution mutations, wherein the amino acid substitution mutations are numbered relative to the LSU of Nicotiana tabacum (SEQ ID NO: 43); and
(b) a Rubisco SSU comprising N8G, K9M, E23D, R28K, V30I, and E88Q amino acid substitution mutations, wherein the amino acid substitution mutations are numbered relative to the S-T2 subunit of Nicotiana tabacum (SEQ ID NO: 44).

12. The genetically engineered plant of claim 11, wherein the Rubisco LSU comprises an amino acid sequence having at least 80%, 85%, 90%, 95%, 98%, or 99% sequence identity to SEQ ID NO: 1.

13. The genetically engineered plant of claim 12, wherein the Rubisco LSU comprises the amino acid sequence of SEQ ID NO: 1.

14. The genetically engineered plant of claim 11, wherein the Rubisco SSU comprises an amino acid sequence having at least 80%, 85%, 90%, 95%, 98%, or 99% sequence identity to SEQ ID NO: 29.

15. The genetically engineered plant of claim 14, wherein the Rubisco SSU comprises the amino acid sequence of SEQ ID NO: 29.

16. A genetically engineered plant comprising:
(a) a Rubisco LSU comprising V91I, V145I, L225I, K429Q, E443D, C449S, V466R, A470E, V472M, and V474T amino acid substitution mutations, wherein the amino acid substitution mutations are numbered relative to the LSU of Nicotiana tabacum (SEQ ID NO: 43); and
(b) a Rubisco SSU comprising N8G, K9M, S22T, E23D, R28K, V30I, N36K, N56H, E88Q, and Q96N amino acid substitution mutations, wherein the amino acid substitution mutations are numbered relative to the S-T2 subunit of Nicotiana tabacum (SEQ ID NO: 44).

17. The genetically engineered plant of claim 16, wherein the Rubisco LSU comprises an amino acid sequence having at least 80%, 85%, 90%, 95%, 98%, or 99% sequence identity to SEQ ID NO: 17.

18. The genetically engineered plant of claim 17, wherein the Rubisco LSU comprises the amino acid sequence of SEQ ID NO: 17.

19. The genetically engineered plant of claim 16, wherein the Rubisco SSU comprises an amino acid sequence having at least 80%, 85%, 90%, 95%, 98%, or 99% sequence identity to SEQ ID NO: 39.

20. The genetically engineered plant of claim 19, wherein the Rubisco SSU comprises the amino acid sequence of SEQ ID NO: 39.

21.-47. (canceled)


SEQUENCE LISTING (44 sequences)

SEQ ID NO: 1 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVQARNEGRDLAQEGNEIIREACKWSPELAAACEVWKEIVFNFAAVDVLDK

SEQ ID NO: 2 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVQARNEGRDLAQEGNEIIREACKWSPELAAACEVWKEIVFNFAAVDVLDK

SEQ ID NO: 3 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEALYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVQARNEGRDLAQEGNEIIREACKWSPELAAACEVWKEIVFNFAAVDVLDK

SEQ ID NO: 4 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDK

SEQ ID NO: 5 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEALFKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDK

SEQ ID NO: 6 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEALFKAQAETGEIKGHYLNATAGTCEEMIKRAVFARELGVPIVMHDYLTGGFTANTTLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAREGNEIIREACKWSPELAAACEVWKEIVFNFAAVDVLDK

SEQ ID NO: 7 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEALYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDK

SEQ ID NO: 8 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEALFKAQAETGEIKGHYLNATAGTCEEMIKRAVFARELGVPIVMHDYLTGGFTANTTLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDK

SEQ ID NO: 9 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEALYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDK

SEQ ID NO: 10 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEALYKAQAETGEIKGHYLNATAGTCEEMIKRAVFARELGVPIVMHDYLTGGFTANTTLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDK

SEQ ID NO: 11 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFCFCAEALYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDK

SEQ ID NO: 12 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFCFCAEALYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNQIIREACKWSPELAAACEVWKEIVFNFAAVDVLDK

SEQ ID NO: 13 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEALYKAQAETGEIKGHYLNATAGTCEEMIKRAVFARELGVPIVMHDYLTGGFTANTTLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAREGNEIIREASKWSPELAAACEVWKEIVFNFAAVDVLDK

SEQ ID NO: 14 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFCFCAEALYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNQIIREASKWSPELAAACEVWKEIVFNFAAVDVLDK

SEQ ID NO: 15 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVQARNEGRDLAQEGNEIIREASKWSPELAAACEVWKEIRFNFEAMDTLDK

SEQ ID NO: 16 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVQARNEGRDLAQEGNDIIREASKWSPELAAACEVWKEIRFNFEAMDTLDK

SEQ ID NO: 17 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVIGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVQARNEGRDLAQEGNDIIREASKWSPELAAACEVWKEIRFNFEAMDTLDK

SEQ ID NO: 18 - protein, 477 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVIGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFIEQDRSRGIYFTQDWVSLPGVLPVASGGIHVWHMPALTEIFGDDSVLQFGGGTLGHPWGNAPGAVANRVALEACVQARNEGRDLAQEGNDIIREASKWSPELAAACEVWKEIRFNFEAMDTLDK

SEQ ID NO: 19 - protein, 480 aa, synthetic construct
MSPQTETKASVGFKAGVKEYKLTYYTPEYQTKDTDILAAFRVTPQPGVPPEEAGAAVAAESSTGTWTTVWTDGLTSLDRYKGRCYRIERVIGEKDQYIAYVAYPLDLFEEGSVTNMFTSIVGNVFGFKALRALRLEDLRIPPAYIKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFIEQDRSRGIYFTQDWVSLPGVLPVASGGIHVWHMPALTEIFGDDSVLQFGGGTLGHPWGNAPGAVANRVALEACVQARNEGRDLAQEGNDIIREASKWSPELAAACEVWKEIRFNFEAMDTLDGEKK

SEQ ID NO: 20 - protein, 123 aa, synthetic construct
MQVWPPIGKKKYETLSYLPDLSEEQLLREIEYLLKNGWVPCLEFETEHGFVYRENNKSPGYYDGRYWTMWKLPMFGCTDATQVLAEVQEAKKAYPQAWIRIIGFDNVRQVQCISFIAYKPEGY

SEQ ID NO: 21 - protein, 123 aa, synthetic construct
MQVWPPYGKKKYETLSYLPDLSEEQLLREIEYLLKNGWVPCLEFETEHGFVYRENNKSPGYYDGRYWTMWKLPMFGCTDATQVLAEVQEAKKAYPQAWIRIIGFDNVRQVQCISFIAYKPEGY

SEQ ID NO: 22 - protein, 123 aa, synthetic construct
MQVWPPYGKKKYETLSYLPDLSEEQLLREIEYLLKNGWVPCLEFETEHGFVYRENNKSPGYYDGRYWTMWKLPMFGCTDATQVLAEVGEAKKAYPQAWIRIIGFDNVRQVQCISFIAYKPEGY

SEQ ID NO: 23 - protein, 123 aa, synthetic construct
MQVWPPYGKKKYETLSYLPDLSEEQLLREIEYLLKNGWVPCLEFETEHGFVYREHNKSPGYYDGRYWTMWKLPMFGCTDATQVLAEVGEAKKAYPQAWIRIIGFDNVRQVQCISFIAYKPEGY

SEQ ID NO: 24 - protein, 123 aa, synthetic construct
MQVWPPINMKKYETLSYLPDLSEEQLLREIEYLLKNGWVPCLEFETEHGFVYRENNKSPGYYDGRYWTMWKLPMFGCTDATQVLAEVGEAKKAYPQAWIRIIGFDNVRQVQCISFIAYKPEGY

SEQ ID NO: 25 - protein, 123 aa, synthetic construct
MQVWPPINMKKYETLSYLPDLSDEQLLKEIEYLLKNGWVPCLEFETEHGFVYRENNKSPGYYDGRYWTMWKLPMFGCTDATQVLAEVGEAKKAYPQAWIRIIGFDNVRQVQCISFIAYKPEGY

SEQ ID NO: 26 - protein, 123 aa, synthetic construct
MQVWPPINMKKYETLSYLPDLSEEQLLREIEYLLKNGWVPCLEFETEHGFVYRENNKSPGYYDGRYWTMWKLPMFGCTDATQVLAEVQEAKKAYPQAWIRIIGFDNVRQVQCISFIAYKPEGY

SEQ ID NO: 27 - protein, 123 aa, synthetic construct
MQVWPPIGMKKYETLSYLPDLSEEQLLREIEYLLKNGWVPCLEFETEHGFVYRENNKSPGYYDGRYWTMWKLPMFGCTDATQVLAEVQEAKKAYPQAWIRIIGFDNVRQVQCISFIAYKPEGY

SEQ ID NO: 28 - protein, 123 aa, synthetic construct
MQVWPPINKKKYETLSYLPDLSEEQLLREIEYLLKNGWVPCLEFETEHGFVYRENNKSPGYYDGRYWTMWKLPMFGCTDATQVLAEVQEAKKAYPQAWIRIIGFDNVRQVQCISFIAYKPEGY

SEQ ID NO: 29 - protein, 123 aa, synthetic construct
MQVWPPIGMKKYETLSYLPDLSDEQLLKEIEYLLKNGWVPCLEFETEHGFVYRENNKSPGYYDGRYWTMWKLPMFGCTDATQVLAEVQEAKKAYPQAWIRIIGFDNVRQVQCISFIAYKPEGY

SEQ ID NO: 30 - protein, 123 aa, synthetic construct
MQVWPPIGKKKYETLSYLPDLSDEQLLKEIEYLLKNGWVPCLEFETEHGFVYRENNKSPGYYDGRYWTMWKLPMFGCTDATQVLAEVQEAKKAYPQAWIRIIGFDNVRQVQCISFIAYKPEGY

SEQ ID NO: 31 - protein, 123 aa, synthetic construct
MQVWPPIGKKKYETLSYLPDLSDEQLLKEIEYLLKNGWVPCLEFETEHGFVYRENNRSPGYYDGRYWTMWKLPMFGCTDATQVLAEVQEAKKAYPQAWIRIIGFDNVRQVQCISFIAYKPEGY

SEQ ID NO: 32 - protein, 123 aa, synthetic construct
MQVWPPINMKKYETLSYLPDLSDEQLLKEIEYLLKNGWVPCLEFETEHGFVYRENNKSPGYYDGRYWTMWKLPMFGCTDATQVLAEVQEAKKAYPQAWIRIIGFDNVRQVQCISFIAYKPEGY

SEQ ID NO: 33 - protein, 123 aa, synthetic construct; identical to SEQ ID NO: 29

SEQ ID NO: 34 - protein, 123 aa, synthetic construct
MQVWPPIGMKKYETLSYLPDLSDEQLLKEIEYLLKNGWVPCLEFETEHGFVYRENNRSPGYYDGRYWTMWKLPMFGCTDATQVLAEVQEAKKAYPQAWIRIIGFDNVRQVQCISFIAYKPEGY

SEQ ID NO: 35 - protein, 123 aa, synthetic construct
MQVWPPINMKKYETLSYLPDLSDEQLLKEIEYLLKNGWVPCLEFETEHGFVYRENNRSPGYYDGRYWTMWKLPMFGCTDATQVLAEVQEAKKAYPQAWIRIIGFDNVRQVQCISFIAYKPEGY

SEQ ID NO: 36 - protein, 123 aa, synthetic construct
MQVWPPINMKKYETLSYLPDLSDEQLLKEIEYLLRNGWVPCLEFETEHGFVYRENNKSPGYYDGRYWTMWKLPMFGCTDATQVLNEVQEAKKAYPQAWIRIIGFDNVRQVQCISFIAYKPEGY

SEQ ID NO: 37 - protein, 123 aa, synthetic construct
MQVWPPINMKKYETLSYLPDLSDEQLLKEIEYLLRNGWVPCLEFETEHGFVYRENNRSPGYYDGRYWTMWKLPMFGCTDATQVLNEVQEAKKAYPQAWIRIIGFDNVRQVQCISFIAYKPEGY

SEQ ID NO: 38 - protein, 123 aa, synthetic construct
MQVWPPINMKKYETLSYLPDLSDEQLLKEIEYLLRSGWVPCLEFETEHGFVYRENNRSPGYYDGRYWTMWKLPMFGCTDATQVLNEVQEAKKAYPQAWIRIIGFDNVRQVQCISFIAYKPEGY

SEQ ID NO: 39 - protein, 123 aa, synthetic construct
MQVWPPIGMKKYETLSYLPDLTDEQLLKEIEYLLKKGWVPCLEFETEHGFVYRENHKSPGYYDGRYWTMWKLPMFGCTDATQVLAEVQEAKKAYPNAWIRIIGFDNVRQVQCISFIAYKPEGY

SEQ ID NO: 40 - protein, 123 aa, synthetic construct
MQVWPPIGKKKYETLSYLPDLTDEQLLKEIEYLLKKGWVPCLEFETEHGFVYRENHKSPGYYDGRYWTMWKLPMFGCTDATQVLAEVQEAKKAYPNAWIRIIGFDNVRQVQCISFIAYKPEGY

SEQ ID NO: 41 - protein, 123 aa, synthetic construct
MQVWPPIGKKKYETLSYLPDLTDEQLLKEIEYLLNKGWVPCLEFETEHGFVYRENHKSPGYYDGRYWTMWKLPMFGCTDATQVLAEVQEAKKAYPNAWIRIIGFDNVRQVQCISFIAYKPEGY

SEQ ID NO: 42 - protein, 123 aa, synthetic construct
MQVWPPIGKKKYETLSYLPDLTDEQLLKEIEYLLNKGWVPCLEFETEHGFVYRENHRSPGYYDGRYWTMWKLPMFGCTDATQVLAEVQEAKKAYPNAWIRIIGFDNVRQVQCISFIAYKPEGY

SEQ ID NO: 43 - protein, 477 aa, Nicotiana tabacum; identical to SEQ ID NO: 7

SEQ ID NO: 44 - protein, 123 aa, Nicotiana tabacum
MQVWPPINKKKYETLSYLPDLSEEQLLREVEYLLKNGWVPCLEFETEHGFVYRENNKSPGYYDGRYWTMWKLPMFGCTDATQVLAEVEEAKKAYPQAWIRIIGFDNVRQVQCISFIAYKPEGY